SEQUENCE LISTING (20 sequences)

SEQ ID NO: 1 - DNA, 691 bp, Homo sapiens
cgggaaggttaaggtaccaaaaatgcaacatcctgaaataaggaggtgttcaaacaatccaggtggcgttcttcattacttggggaccagatgtgctgtgacaattgtgctcaggtgattgaagtgacacccaggtcatatatacccagggtggaggggttctggggtccttcatttgaagtgtgatatgggacaagagcagaggagactccatccaccctagccagctttcctgagacttgaggaccaacttgacatgaatcctaggcttctgcttatctttgatgcctcactgtgagtagtagacctgctttatgtaacttgtgattgttttgtctcatcagatttatgcaattgggagagatactggggttcctctttggctcctctctactgtcttcattatgttagaatgactgcagcagccagttctactctaagcccccactaaacttgtgaacctttgcaagaagctactgggataagtgacttttgcaaaatttcaagatatgacatcaatatacaaatatcaattatactatatctttaacaataaatagcaagaaaattgatttaaaagtaatattttcatagaataaaaatagaatttgctttgagacagatataacagaatatacgcaagatctgcacatttaaaactatgaaaaattgctgacagtatttaaagatc

SEQ ID NO: 2 - DNA, 580 bp, Homo sapiens
ctatgtctcacagtccagacttggagtacaagtaataagaagaataaaacttgatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaaaaaaaaaaaaacctatagtgagtcgtattaattcggatccgc

SEQ ID NO: 3 - DNA, 20 bp, artificial
  Forward primer CENPF exon 6
gtgttctcatggcagcaaga

SEQ ID NO: 4 - DNA, 24 bp, artificial
  Reverse primer exon 11 CENPF
ctgtttgatgttcttgagttctgc

SEQ ID NO: 5 - DNA, 19 bp, artificial
  RCC2 primer
tgcgtttgctggctttgat

SEQ ID NO: 6 - DNA, 17 bp, artificial
  ARFGEF exon 1 primer
tagccgacaaggtgaag

SEQ ID NO: 7 - DNA, 20 bp, artificial
  SULF2 exon 6 primer
gtgtagcgcatgatccagtg

SEQ ID NO: 8 - DNA, 19 bp, artificial
  RPS6KB1 forward primer
gctgaactttaggagccag

SEQ ID NO: 9 - DNA, 20 bp, artificial
  TMEM49 reverse primer
ttttcctcccaagcaaaaca

SEQ ID NO: 10 - DNA, 21 bp, artificial
  3′RACE primer exon 3 of ATXN7
ctgaagtgatgctgggacagt

SEQ ID NO: 11 - DNA, 23 bp, artificial
  3′ RACE primer from exon 4 of ATXN7
acagaattggacgaaagtttcaa

SEQ ID NO: 12 - DNA, 23 bp, artificial
  5′ RACE primers from exon 12 of ATXN7
acagaattggacgaaagtttcaa

SEQ ID NO: 13 - DNA, 23 bp, artificial
  5′ RACE nested primer  from exon12
atttgctggatttcaatttctga

SEQ ID NO: 14 - DNA, 23 bp, artificial
  MTAP primer exon 4
atcatgccttcaaaggtcaacta

SEQ ID NO: 15 - DNA, 287 bp, Homo sapiens
cgcggatccagacgctgcgtttgctggctttgatgaaatgcacaacgtcctgcaggctgaactggataaactcacatcagtaaagcaacagctagaaaacaatttggaagagtttaagcaaaagttgtgcagagctgaacaggcgttccaggcgagtcagatcaaggagaatgagctgaggagaagcatggaggaaatgaagaaggaaaacaacctccttaagagtcactctgagcaaaaggccagagaagtctgccacctggaggcagaatcaagaacatcaaata

SEQ ID NO: 16 - DNA, 641 bp, Homo sapiens
gctcggcgtgatgtgctgagatgcgtttgggaagaggcgtgaatattgtggggctgaatcctcagggccgtggggggctgcatggctgatgaccatgaggactggcctgtgcgggtacatcttcttggacgtgcggaagaagctcacgctgtcattggtgatgaggtctgtgaggtaatccttggagtagtcggagccgtgcttctctttcaccccgttccgacacagcgtgtagttataaaagcgggagtttttaaggagtccgacccactccttccagccgggtggcacgtaggagccgttgtattcattaagatacttcccgaagaaagctgtccggtagccagtgctattgaggtacacggcaaaggtgcggctctcgtgctgtgcctgccgggagggcgaggagcagttctcattgttggtgtaggtgttgtggttgtggacgtacttgccggtgaggatggaggagcgtgaggggcagcacatgggtgtggtcacgaaggcgttgatgaagtgcgtcccgccctgctccatgatgcgccgggtcttgttcatcacctgcatggaaccgagcgccacctggcaggccctgcgcagctgggagtgctggggccgcttcacctccttgtcggctagga

SEQ ID NO: 17 - DNA, 786 bp, Homo sapiens
agacagggaagctgaggacatggcaggagtgtttgacatagacatagacctggaccagccagaggacgcgggctctgaggatgagctggaggaggggggtcagttaaatgaaagcatggaccatgggggagttggaccatatgaacttggcatggaacattgtgagaaatttgaaatctcagaaactagtgtgaacagagggccagaaaaaatcagaccagaatgttttgagctacttcgggctgggaaaatatttgccatgaaggtgcttaaaaagggagaaaactggttgtcctggatgtttgaaaagttgaactcagaggagaaaactaaataagtagagaaagttttaactgcagaaattggagtggatgggttctgccttaaattgggaggactccaagctgggaaggaaaattcccttttccaacctgtatcaatttttacaacttttttcctgaaaagcagtttagtccatactttgcactgacatactttttccttctgtgctaaggtaaggtatccaccctcggatgcaatccaccttgtgttttcttagggtggaatgtgatgttcagcagcaaacttgcaacagactggccttctgtttgttactttcaaaaggcccacatgatacaattagagaattcatcaaaatgtatataaattatctagattggataacagtcttgcatgtttatcatgttacaatttaatattccatcctgcccaacccttcctctcccatcctcaaaaagggccattttatgatgcattgcacaccct

SEQ ID NO: 18 - DNA, 751 bp, Homo sapiens
cagaattggacgaaagtttcaaggagtttgggaaaaaccgcgaagtcatggggctctgttcgggaaggttaaggtaccaaaaatgcaacatcctgaaataaggaggtgttcaaacaatccaggtggcgttcttcattacttggggaccagatgtgctgtgacaattgtgctcaggtgattgaagtgacacccaggtcatatatacccagggtggaggggttctggggtccttcatttgaagtgtgatatgggacaagagcagaggagactccatccaccctagccagctttcctgagacttgaggaccaacttgacatgaatcctaggcttctgcttatctttgatgcctcactgtgagtagtagacctgctttatgtaacttgtgattgttttgtctcatcagatttatgcaattgggagagatactggggttcctctttggctcctctctactgtcttcattatgttagaatgactgcagcagccagttctactctaagcccccactaaacttgtgaacctttgcaagaagctactgggataagtgacttttgcaaaatttcaagatatgacatcaatatacaaatatcaattatactatatctttaacaataaatagcaagaaaattgatttaaaagtaatattttcatagaataaaaatagaatttgctttgagacagatataacagaatatacgcaagatctgcacatttaaaactatgaaaaattgctgacagtatttaaagatc

SEQ ID NO: 19 - DNA, 818 bp, Homo sapiens
tttgctggatttcaatttctgaggtttcctggacatgggggaggaaggaaccgaggaaaggccagagggcgtggaaggggatgaggatgaagaggacacttgtctggattgcatactgcacacaggatccatcgcccctgaagcagcaggctgtgcatttagtgtgtttccatgagctggtaccgatttgctatttggggagatgcaggtagatgagagcaggactggggatgtagagacggtggctgctgccagatagctgactccacattgtgatgtcggcacagagtttgtccggtgaggaatacgtgtggagatgggtgaggtggtactgggcactggtgggattttccaaactgtggagcaggcaagattttagccgctcgaattgggccatgtcggacagagaagagctcttgtgcttcgccactgatagggatgctccagacctgcattccatcactgtagccaatcataatcaacaaaggcggttcactcccagtactatgtatttcatgaaattccagatttcttgatgtatcatttaaatctgcattttcaaatctgacccagactattttctccttttcttctgttagaggtgttccactgtaagcctgtggcacaacatcctgcagaaaagtcacaacactttccatgtaggactgctctgtgacagcctgggggcgaaccacaactccaccagtacaacgactgggtcttcttggggaatctgtagccatagcttcattcataaaaccggccgccccgccgttaactttcatcaaagccagcaaacgcagtgttcggatccgcga

SEQ ID NO: 20 - DNA, 728 bp, Homo sapiens
tcatgccttcaaaggtcaactaccaggcgaacatctgggctttgaaggaagagggctgtacacatgtcatagtgaccacagcttgtggctccttgagggaggagattcagcccggcgatattgtcattattgatcagttcattgacagctatgtctcacagtccagacttggagtacaagtaataagaagaataaaacttgatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaaaaaaaaaaaaacctatagtgagtcgtattaattcggatccgc

(19) United States
(12) Patent Application Publication
(10) Pub. No.: US 2010/0285475 A1
(43) Pub. Date: Nov. 11, 2010

(54) FUSED GENES

(75) Inventors: Nallasivam Palanisamy, Singapore (SG); Kalpana Ramnarayanan, Singapore (SG); Edison T. Liu, Singapore (SG)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: AGENCY FOR SCIENCE, TECHNOLOGY AND RESEARCH, Singapore (SG)

(21) Appl. No.: 12/739,090

(22) PCT Filed: Oct. 22, 2007
(86) PCT No.: PCT/SG2007/000361

§ 371 Date: Apr. 21, 2010

(51) Int. Cl. C12Q 1/68; C07H 21/04; C12N 15/63; C40B 40/06; C40B 30/00

(52) U.S. Cl. 435/6; 536/23.1; 435/320.1; 506/16; 506/7

(57) ABSTRACT

There is provided at least one isolated fused gene comprising at least one first gene and/or fragment thereof fused to at least one second gene and/or fragment thereof, wherein at least the first and/or the second gene, independently, is selected from the group consisting of: RCC2, CENPF, ARFGEF2, SULF2, MTAP, ATXN7, BCAS3, RPS6KB1, TMEM49, EAP30, a gene having the nucleotide sequence SEQ ID NO:1, and a gene having the nucleic acid SEQ ID NO:2, or a fragment thereof. There is also provided a diagnostic method and/or a kit for detecting the susceptibility, prognosis, and/or to tumour in a subject.

### FIELD OF THE INVENTION

The present invention relates to isolated fused gene implicated in tumour, in particular breast tumour. The invention also provides a kit for the detection of the fused genes for the diagnosis and/or prognosis of tumour in a subject.

### BACKGROUND OF THE ART

Chromosomal aberrations including deletions, duplications, inversions, insertions and translocations are the characteristic feature of many cancer types. Primary focus of cancer genome analysis is to identify genes that are perturbed and play a role in cancer development. Many deregulated and fusion genes have been identified by cloning breakpoint junctions of chromosome translocations in hematological malignancies and soft tissue sarcomas. Chromosome translocations can cause deregulation of genes at the breakpoints which result in neoplastic transformation. There are two major molecular consequences associated with chromosome translocations; first, the promoter and/or enhancer element of a gene is placed near an oncogene result in over expression of the oncogene. Secondly, formation of a fusion gene produced by breakage and joining within introns of two genes result in expression of a fusion protein.

Among the different types of chromosome aberrations, recurrent translocations are prevalent and well characterized in hematological malignancies. In many solid tumor cancers, despite the presence of many structural aberrations, mostly unbalanced translocations, tumor specific recurrent translocations are difficult to characterize due to several technical limitations with the available technologies. A recently cloned recurrent fusion gene in prostate cancer, using bioinformatics analysis of gene expression microarray data (Tomlins et al., 2005), set a new paradigm shift towards understanding the molecular complexity in solid tumors.

The most common problem in solid tumor cancer genome analysis is the failure to characterize unbalanced copy number changes and complex rearrangements. Gene expression micro array and low-resolution copy number analysis methods do not provide information on genomic rearrangements. Conventional cytogenetic karyotyping analysis on hematological malignancies and solid tumors identified 52,172 (http://cgap.nci.nih.gov/Chromosomes/Mitelman) abnormal karyotypes as on May 16, 2007. Complete molecular characterization of various chromosome rearrangements resulted in the identification of more than 358 fusion genes (Mitelman et al., 2007). Specificity of chromosome translocations lead to sub classification of tumors solely based on chromosome aberrations. Until date, about 500 such tumor specific translocations are identified. In spite of the higher incidence of cancer death due to solid tumor cancer (80%) when compared with hematological malignancies (10%) the proportion of available cytogenetics information, appear to be more in hematological malignancies. The cytogenetic changes in hematological malignancies are very few even in advanced stage cancers and the type of chromosome changes are specific to particular histological type. Chromosome aberrations in solid tumors are highly complex even at the early stage or at diagnosis making it impossible for the correct identification of all abnormal chromosomes. Among the various changes the distinction between tumor associated primary abnormality and progression associated changes are not possible. Additional complexities are due to clonal heterogeneity, which is present in less than 5% of hematological cancers but very common in solid tumors.

Among many types of solid tumors, breast cancer is one of the tumor types for which the chromosome abnormalities are not well studied. According to recent estimates from American Cancer Society; about 212,920 women will be diagnosed and 40,970 are predicted to have died of breast cancer in the year 2006 (ACS, 2006). Current understanding on the genetic basis of breast cancer is limited to mutated and amplified genes in a proportion of breast cancer patients. Breast cancer genome is characterized by the presence of highly unbalanced aneuploial karyotype with complex structural rearrangements and numerical aberrations. It is evident from the literature review that identification of recurrent aberrations is nearly impossible with currently available cytogenetic and molecular methods.

Although cloning of fusions genes by molecular characterization of chromosome translocation identified by G-band karyotyping has been a successful approach in hematological malignancies and soft tissue sarcomas, the highly complex genomic rearrangements and identification of recurrent chromosome translocations by G-band karyotyping is often difficult due to poor chromosome morphology and clonal heterogeneity in solid tumors. As evident from the MCF7 data more than 60% of copy number boundaries are located within known genes that can be directly selected for further validation.

To date, no recurrent translocation producing fusion genes have been identified in breast cancer and the current invention provides a new approach to identify fusion genes based on the analysis of unbalanced copy number changes

### SUMMARY OF THE INVENTION

The present invention addresses the problems above, and in particular to provides new and/or improved use of the CGH method for the identification of copy number transition (CNT) regions comprising the fused genes therein. The invention also provides the use of novel fused genes identified in the invention as biomarkers in the diagnosis of solid tumours.

According to one aspect of the current invention, there is provided an isolated fused gene comprising at least one first gene and/or fragment thereof fused to at least one second gene and/or fragment thereof. The at least one first and/or the second gene may independently, be selected from the group of genes consisting of: RCC2, CENPF, ARFGEF2, SULF2, MTAP, ATXN7, BCAS3, RPS6 KB1, TMEM49, EAP30, a gene having the nucleotide sequence SEQ ID NO:1, and a gene having the nucleic acid SEQ ID NO:2, or a fragment thereof. The fusion of the genes may be by genomic translocation, insertion, inversion, amplification and/or deletion. The fused gene may be selected from the group of fused genes: RCC2/CENPF, ARFGEF2/SULF2, MTAP/a gene comprising the nucleotide sequence SEQ ID NO:2, ATXN7/a gene comprising the nucleotide sequence SEQ ID NO:1, BCAS3/ATXN7, RPS6 KB1/TMEM49, and RPS6 KB1/EAP30, or fragment(s) thereof. A non-exclusive list of fused genes according to the invention is summarised in Table 1. In particular, one fused gene according to the invention is ARFGEF2/SULF2 fusion gene comprising the nucleic acid sequence of SEQ ID NO: 16 and/or a fragment thereof. Another fused gene according to the invention is RPS6 KB1/TMEM49 fusion gene comprising the nucleic acid sequence of SEQ ID NO: 17 and/or a fragment thereof. Another fused gene according to the invention is ATXN7/a gene having the nucleotide sequence SEQ ID NO:1. This fused gene comprises the nucleic acid sequence of SEQ ID NO: 18 and/or a fragment thereof. Another fused gene according to the invention is ATXN7/BCAS3 fusion gene comprising the nucleic acid sequence of SEQ ID NO: 19 and/or a fragment thereof. The fused may also be MTAP /a gene having the nucleotide sequence SEQ ID NO:2, the gene fusion comprising the nucleic acid sequence of SEQ ID NO: 20 and/or a fragment thereof. Any of the fused gene(s) may be comprised in a vector.

According to another aspect of the current invention, there is provided an isolated nucleic acid comprising the nucleotide sequence SEQ ID NO:1 and/or SEQ ID NO:2, or a fragment thereof. The isolated nucleic acid may be comprised in a vector.

According to yet another aspect of the invention there is also provided a diagnostic and/or prognostic kit for the diagnosis and/or prognosis of tumour in a subject comprising detecting at least one fused gene, wherein the presence of the fused gene is indicative of presence and/or the stage of tumour.

The diagnostic and/or prognostic kit may comprise at least one nucleic acid molecule capable of hybridizing to and/or complementary to the fused gene and/or a fragment thereof, wherein hybridization is indicative of presence and/or the stage of tumour.

The invention further provides a diagnostic and/or prognostic kit for the diagnosis and/or prognosis of tumour in a subject, wherein the kit comprises one or more fragment representative of a genome capable of hybridizing to differentially labelled genomic DNA isolated from tumour tissue from at least one subject and from a control tissue, wherein an increase or decrease of the hybridization intensity and/or signal(s) of the label in the tumour tissue, compared to that in control tissue detects copy number transition (CNT) regions in the tumour tissue, indicative of presence and/or stage of tumour.

The CNT regions detected by the diagnostic and/or prognostic kit may comprise fused gene(s).

The fused gene detected in the diagnosis and/or prognosis of tumour in a subject may be selected from the group of fused genes: RCC2/CENPF, ARFGEF2/SULF2, MTAP/a gene comprising the nucleotide sequence SEQ ID NO:2, ATXN7/a gene comprising the nucleotide sequence SEQ ID NO:1, BCAS3/ATXN7, RPS6 KB1/TMEM49, and RPS6 KB1/EAP30, or fragment(s) thereof.

The fused genes may further be detected by fluorescence in situ hybridization (FISH) and/or rapid amplification of cDNA end polymerase chain reaction (RACE-PCR) technique. The tumour may be stage III tumour. In particular the tumour may be solid tumour. More in particular the tumour may be breast tumour.

According to a further aspect of the invention, there is provided a method of diagnosis and/or prognosis of presence and/or stage of tumour in a subject comprising detecting at least one fused gene, wherein the presence of the fused gene is indicative of presence and/or the stage of tumour.

The method may comprise providing at least one nucleic acid molecule capable of hybridizing to and/or complementary to the fused gene and/or a fragment thereof, wherein hybridization is indicative of presence and/or the stage of tumour.

According to yet another aspect there is also provided a method of diagnosis and/or prognosis of presence and/or stage of tumour in a subject, wherein the method comprises providing one or more fragments representative of a genome capable of hybridizing to differentially labelled genomic DNA isolated from tumour tissue from at least one subject and from a control tissue, wherein an increase or decrease of the hybridization intensity and/or signal(s) of the label in the tumour tissue, compared to that in control tissue detects copy number transition (CNT) regions in the tumour tissue, indicative of presence and/or stage of tumour.

The CNT regions may comprise any fused gene(s) according to the invention. The fused gene detected in the diagnosis and/or prognosis of tumour in a subject may be selected from the group of fused genes: RCC2/CENPF, ARFGEF2/SULF2, MTAP/a gene comprising the nucleotide sequence SEQ ID NO:2, ATXN7/a gene comprising the nucleotide sequence SEQ ID NO:1, BCAS3/ATXN7, RPS6 KB1/TMEM49, and RPS6 KB1/EAP30, or fragment(s) thereof.

The fused genes may further be detected by FISH and/or RACE technique. The tumour may be stage III tumour. In particular, the tumour may be solid tumour. More in particular, the tumour may be breast tumour.

There is further provided a kit for the detecting the presence of fused genes, wherein the kit comprises one or more fragments representative of a genome capable of hybridizing to differentially labelled control and test, genomic DNA wherein an increase or decrease of the hybridization intensity and/or signal(s) of test genome, compared to that in control genome detects copy number transition (CNT) regions in the test genome, wherein the CNT regions comprise fused genes.

According to yet another aspect the invention provides a method of detecting the presence of fused genes, wherein the method comprises providing one or more fragments representative of a genome capable of hybridizing to differentially labelled control and test genomic DNA wherein an increase or decrease of the hybridization intensity and/or signal(s) of test genome, compared to that in control genome detects copy number transition (CNT) regions in the test genome, wherein the CNT regions comprise fused genes.

The fused gene detected in the diagnosis and/or prognosis of tumour in a subject may be selected from the group of fused genes: RCC2/CENPF, ARFGEF2/SULF2, MTAP/a gene comprising the nucleotide sequence SEQ ID NO:2, ATXN7/a gene comprising the nucleotide sequence SEQ ID NO:1, BCAS3/ATXN7, RPS6 KB1/TMEM49, and RPS6 KB1/EAP30, or fragment(s) thereof.

### BRIEF DESCRIPTION OF THE FIGURES

FIG. 1: CGH array method. Hybridization of tumour and reference DNA to oligo array, image scanning and ratio profile analysis provide regions of unbalanced copy number changes.

FIG. 2. (A): Identification of a CNT locus. (B) Comparison of 44K, 185K and 244 k array designs.

FIG. 3: Spectral karyotype analysis, of MCF7 genome and identification of many structural unbalance rearrangements.

FIG. 4: Isolation of fusion gene from a region of copy number transition region.

FIG. 5: Validation of CNT region in CENPF gene. (A) a-CGH profile of chromosome 1 and identification of a region CNT region at 1q41. (B) High resolution view showing CNT region within 10,827 bp. Green or Light grey and red or dark grey vertical bars indicate the location of BAC clones from the 5′ and 3′ of CENPF gene showing loss and gain respectively. (C) Spectral karyotyping showing the genomic organization of chromosome 1 in MCF7. (D) Confirmation of rearrangement by FISH, two normal signals (co localized red or dark grey and green or light grey signals-Light grey arrows) and three red or dark grey signals on different chromosomes (white arrows).

FIG. 6: (A) Genomic organization of CENPF gene, the CNT locus shown in dotted box. Arrows indicate the direction of RACE PCR. (B) 3′ and 5′ RACE PCR showing a 270 by amplified product in 5′RACE. (C) Gene expression analysis in treated and untreated cells with triplicate experiments for each time point. (D) Sequence of PCR product show exons 9, 10 and 11 and 46 by sequence from RCC2 showing RCC2/CENPF (SEQ ID NO: 15).

FIG. 7: RT PCR validation of CENPF in breast cancer cell lines.

FIG. 8: RT PCR validation of CENPF in primary breast cancer tumors.

FIG. 9: Expression of normal CENPF transcript in primary breast cancer tumors.

FIG. 10: FISH analysis of an amplified region on 17q23 showing insertion of the amplified sequences in multiple locations in MCF7 genome. (A) Interphase nuclei. (B) Metaphase chromosomes.

FIG. 11: (A) 10 mb region of amplification showing many CNT within genes. (B) Inversion of 1.1 mb region within ARFGEF2 and SULF2 genes. (C) A 2.7 kb PCR product amplified by 3′RACE PCR. (D) Sequence of ARFGEF2 and SULF2 fusion gene (SEQ ID NO: 16).

FIG. 12: FISH analysis of MCF7 showing amplification and fusion of ARFGEF2 and SULF2 genes. (A) metaphase chromosome. (B) Interphase nuclei.

FIG. 13: (A, B) RT PCR analysis of ARFGF2/SULF2 fusion gene in breast cancer tumors.

FIG. 14: (A) BLAST search showing alignment of SULF2 sequence to exons 3-6. (B) Variant fusion gene skipping exon 5 in SULF2 gene. (C) Alignment with first exon of ARFGEF2.

FIG. 15: FISH analysis using BAC RP11-111G18 shows high-level amplification of RPS6 KB1 gene in MCF7.

FIG. 16A: A. 17q23 amplicon with CNT regions in genes.

FIG. 16B: 3′ RACE PCR amplified a 1.2 kb PCR product. Lane1shows the product following a HindIII digest, lanes 3-6 show amplification product in cell lines CCL159 (lane 3), MCF7 (lane 4), MCF10 (lane 5) and HCT116 (lane 6).

FIG. 17: 3′RACE PCR from RPS6 Kb1 amplified normal transcript in all cell line and a small band in BT474 cell line.

FIG. 18: Metaphase FISH analysis showing fusion of RPS6 Kb1 (white spots) and EAP30 (/light grey) genes.

FIG. 19: (A) Differential amplification of 5′ and 3′ segment of ATXN7 gene forming two CNT regions. (B) BCAS3 gene with two CNT regions.

FIG. 20: (A) FISH analysis using BAC 1143K18 showing the amplification and insertion of ATXN7 gene sequences at multiple locations in MCF7. (B) 3′ and 5′ RACE from the two CNT regions amplified distinct PCR products. (C) FISH analysis showing fusion of ATXN7 and BCAS3 gene at on chromosome 1p21. (D,) Fusion gene sequence of ATXN7 and novel gene of SEQ ID NO: 1 (SEQ ID NO: 18) (E) BLAST search alignment for ATXN7 and Novel gene, (F, G) BLAST search alignment for BCAS3;ATXN7.

FIG. 21: (A) aCGH identified deletion of 254 kb region with variable copy number due to clonal heterogeneity of deletion in MCF7. (B). 3′ RACE PCR showing amplification of 728 by product. (C) Illustration showing the genomic organization of MTAP gene with a CNT region in intron 4. (D) Gene expression analysis shows no expression for all the genes including MTAP. (E). Genomic organization of the deleted region on 9p21. BLAST search shows the fusion of exon 4 of MTAP fused with an EST from the immediately flanking region of the deletion. (F) Sequence of MTAP/EST of SEQ ID NO: 2 fusion gene (SEQ ID NO: 20).

### BRIEF DESCRIPTION OF SEQUENCES

SEQ ID NO: 1: Novel gene:
5′CGGGAAGGTTAAGGTACCAAAAATGCAACATCCTGAAATAAGGAGGTGTTCA
AACAATCCAGGTGGCGTTCTTCATTACTTGGGGACCAGATGTGCTGTGACAATTGTGC
TCAGGTGATTGAAGTGACACCCAGGTCATATATACCCAGGGTGGAGGGGTTCTGGGG
TCCTTCATTTGAAGTGTGATATGGGACAAGAGCAGAGGAGACTCCATCCACCCTAGCC
AGCTTTCCTGAGACTTGAGGACCAACTTGACATGAATCCTAGGCTTCTGCTTATCTTTG
ATGCCTCACTGTGAGTAGTAGACCTGCTTTATGTAACTTGTGATTGTTTTGTCTCATCA
GATTTATGCAATTGGGAGAGATACTGGGGTTCCTCTTTGGCTCCTCTCTACTGTCTTCA
TTATGTTAGAATGACTGCAGCAGCCAGTTCTACTCTAAGCCCCCACTAAACTTGTGAAC
CTTTGCAAGAAGCTACTGGGATAAGTGACTTTTGCAAAATTTCAAGATATGACATCAAT
ATACAAATATCAATTATACTATATCTTTAACAATAAATAGCAAGAAAATTGATTTAAAAGT
AATATTTTCATAGAATAAAAATAGAATTTGCTTTGAGACAGATATAACAGAATATACGCA
AGATCTGCACATTTAAAACTATGAAAAATTGCTGACAGTATTTAAAGATC3′
SEQ ID NO: 2: Novel gene:
5′CTATGTCTCACAGTCCAGACTTGGAGTACAAGTAATAAGAAGAATAAAACTTG
ATCCCTTAAGTAGATTCACCATAAGTTAGCTCAGAGCAATTCCAGTGCAAGTATGGTCT
GTGATCCAGTAGTATCTTACAGACAGCAAGTTGAACATTGTGGGATGCATGAGCTATT
GAGGCCTTTGCAGCTTTCTGCTACATGGAGGCTAGGGCCAGAGTCAAGATTTATGCTT
TGCAGCACACTGGTCAGCTGTTTTTGCAAATCAGATTAAATGATTTTTAAATGAGGCTG
AGAGCATGGGAGATACTAATGTGTGTTTCCTTGTGAGCTACTGCATAAGTTAGGAAATT
GAAATACAGAAAGATGAAAAGTGATTTGCCCAAGCATATAGATCAAAGCTGTGGCAGA
ACCAGGACTGGAACCTATATCTCTCTACTAATGGTTTTTTTAAAAAAATAACCTTGTTTC
AAAAATATTAAAAAGTCACAAGAAAGGTAAACATGTGGATAAACAAAATGAAGAAAATA
AAAATTATCCAGTAAAAAAAAAAAAACCTATAGTGAGTCGTATTAATTCGGATCCGC3′
SEQ ID NO: 3: CENPF exon 6 primer sequence:
5′ GTGTTCTCATGGCAGCAAGA 3′
SEQ ID NO: 4: CENPF exon 6 primer sequence:
5′CTGTTTGATGTTCTTGAGTTCTGC3′
SEQ ID NO: 5: RCC2 primer sequence: 5′ TGCGTTTGCTGGCTTTGAT3′
SEQ ID NO: 6: ARFGEF2 exon 1 primer sequence:
5′ TAGCCGACAAGGTGAAG 3′
SEQ ID NO: 7: ARFGEF2 exon 6 primer sequence:
5′ GTGTAGCGCATGATCCAGTG 3′
SEQ ID NO: 8: RPS6KB1 forward primer: 5′GCTGAAC TTTAGGAGCCAG3′
SEQ ID NO: 9: TMEM49 reverse primer: 5′TTTTCCTCCCAAGCAAAACA3′
SEQ ID NO: 10: ATXN7 exon 3 primer 3′ RACE primer:
5′CTGAAGTGATGCTGGGACAGT3′
SEQ ID NO: 11: ATXN7 exon 4 nested 3′ RACE primer:
5′ACAGAATTGGACGAAAGTTTCAA3′
SEQ ID NO: 12: ATXN7 exon 12 primer 5′ RACE primer:
5′GGTACTGCTACTGGCATTTTGAC3′
SEQ ID NO: 13: ATXN7 exon 12 primer 5′ nested RACE primer:
5′ATTTGCTGGATTTCAATTTCTGA3′
SEQ ID NO: 14: MTAP exon 4 primer: 5′ATCATGCCTTCAAAGGTCAACTA3′
SEQ ID NO: 15: Sequence of RCC2/ CENPF fusion gene. RCC2 sequence
(underlined) fused to CENPF sequence:
5′CGCGGATCCAGACGCTGCGTTTGCTGGCTTTGATGAAATGCACAACGTCCT
GCAGGCTGAACTGGATAAACTCACATCAGTAAAGCAACAGCTAGAAAACAATTTGGAA
GAGTTTAAGCAAAAGTTGTGCAGAGCTGAACAGGCGTTCCAGGCGAGTCAGATCAAG
GAGAATGAGCTGAGGAGAAGCATGGAGGAAATGAAGAAGGAAAACAACCTCCTTAAG
AGTCACTCTGAGCAAAAGGCCAGAGAAGTCTGCCACCTGGAGGCAGAATCAAGAACA
TCAAATA3′
SEQ ID NO: 16: Sequence of SULF2 / ARFGEF2 fusion gene. SULF2
sequence (underlined) fused to ARFGEF2 sequence:
5′GCTCGGCGTGATGTGCTGAGATGCGTTTGGGAAGAGGCGTGAATATTGTGG
GGCTGAATCCTCAGGGCCGTGGGGGGCTGCATGGCTGATGACCATGAGGACTGGCC
TGTGCGGGTACATCTTCTTGGACGTGCGGAAGAAGCTCACGCTGTCATTGGTGATGA
GGTCTGTGAGGTAATCCTTGGAGTAGTCGGAGCCGTGCTTCTCTTTCACCCCGTTCCG
ACACAGCGTGTAGTTATAAAAGCGGGAGTTTTTAAGGAGTCCGACCCACTCCTTCCAG
CCGGGTGGCACGTAGGAGCCGTTGTATTCATTAAGATACTTCCCGAAGAAAGCTGTCC
GGTAGCCAGTGCTATTGAGGTACACGGCAAAGGTGCGGCTCTCGTGCTGTGCCTGCC
GGGAGGGCGAGGAGCAGTTCTCATTGTTGGTGTAGGTGTTGTGGTTGTGGACGTACT
TGCCGGTGAGGATGGAGGAGCGTGAGGGGCAGCACATGGGTGTGGTCACGAAGGCG
TTGATGAAGTGCGTCCCGCCCTGCTCCATGATGCGCCGGGTCTTGTTCATCACCTGCA
TGGAACCGAGCGCCACCTGGCAGGCCCTGCGCAGCTGGGAGTGCTGGGGCCGCTTC
ACCTCCTTGTCGGCTAGGA3′
SEQ ID NO: 17: Sequence of RPS6KB1 / TMEM49 fusion gene. RPS6KB1
sequence (underlined) fused to TMEM49 sequence:
5′AGACAGGGAAGCTGAGGACATGGCAGGAGTGTTTGACATAGACATAGACCT
GGACCAGCCAGAGGACGCGGGCTCTGAGGATGAGCTGGAGGAGGGGGGTCAGTTAA
ATGAAAGCATGGACCATGGGGGAGTTGGACCATATGAACTTGGCATGGAACATTGTGA
GAAATTTGAAATCTCAGAAACTAGTGTGAACAGAGGGCCAGAAAAAATCAGACCAGAA
TGTTTTGAGCTACTTCGGGCTGGGAAAATATTTGCCATGAAGGTGCTTAAAAAGGGAG
AAAACTGGTTGTCCTGGATGTTTGAAAAGTTGAACTCAGAGGAGAAAACTAAATAAGTA
GAGAAAGTTTTAACTGCAGAAATTGGAGTGGATGGGTTCTGCCTTAAATTGGGAGGAC
TCCAAGCTGGGAAGGAAAATTCCCTTTTCCAACCTGTATCAATTTTTACAACTTTTTTCC
TGAAAAGCAGTTTAGTCCATACTTTGCACTGACATACTTTTTCCTTCTGTGCTAAGGTA
AGGTATCCACCCTCGGATGCAATCCACCTTGTGTTTTCTTAGGGTGGAATGTGATGTT
CAGCAGCAAACTTGCAACAGACTGGCCTTCTGTTTGTTACTTTCAAAAGGCCCACATG
ATACAATTAGAGAATTCATCAAAATGTATATAAATTATCTAGATTGGATAACAGTCTTGC
ATGTTTATCATGTTACAATTTAATATTCCATCCTGCCCAACCCTTCCTCTCCCATCCTCA
AAAAGGGCCATTTTATGATGCATTGCACACCCT3′
SEQ ID NO: 18: Sequence of ATXN7 / novel gene of SEQ ID NO: 1. ATXN7
sequence (underlined) fused to novel gene of SEQ ID NO: 1:
5′CAGAATTGGACGAAAGTTTCAAGGAGTTTGGGAAAAACCGCGAAGTCATGG
GGCTCTGTTCGGGAAGGTTAAGGTACCAAAAATGCAACATCCTGAAATAAGGAGGTGT
TCAAACAATCCAGGTGGCGTTCTTCATTACTTGGGGACCAGATGTGCTGTGACAATTG
TGCTCAGGTGATTGAAGTGACACCCAGGTCATATATACCCAGGGTGGAGGGGTTCTG
GGGTCCTTCATTTGAAGTGTGATATGGGACAAGAGCAGAGGAGACTCCATCCACCCTA
GCCAGCTTTCCTGAGACTTGAGGACCAACTTGACATGAATCCTAGGCTTCTGCTTATC
TTTGATGCCTCACTGTGAGTAGTAGACCTGCTTTATGTAACTTGTGATTGTTTTGTCTC
ATCAGATTTATGCAATTGGGAGAGATACTGGGGTTCCTCTTTGGCTCCTCTCTACTGTC
TTCATTATGTTAGAATGACTGCAGCAGCCAGTTCTACTCTAAGCCCCCACTAAACTTGT
GAACCTTTGCAAGAAGCTACTGGGATAAGTGACTTTTGCAAAATTTCAAGATATGACAT
CAATATACAAATATCAATTATACTATATCTTTAACAATAAATAGCAAGAAAATTGATTTAA
AAGTAATATTTTCATAGAATAAAAATAGAATTTGCTTTGAGACAGATATAACAGAATATA
CGCAAGATCTGCACATTTAAAACTATGAAAAATTGCTGACAGTATTTAAAGATC3′
SEQ ID NO: 19: Sequence of ATXN7 / BCAS3 fusion gene. ATXN7 sequence
(underlined) fused to BCAS3 sequence:
5′TTTGCTGGATTTCAATTTCTGAGGTTTCCTGGACATGGGGGAGGAAGGAACC
GAGGAAAGGCCAGAGGGCGTGGAAGGGGATGAGGATGAAGAGGACACTTGTCTGGA
TTGCATACTGCACACAGGATCCATCGCCCCTGAAGCAGCAGGCTGTGCATTTAGTGTG
TTTCCATGAGCTGGTACCGATTTGCTATTTGGGGAGATGCAGGTAGATGAGAGCAGGA
CTGGGGATGTAGAGACGGTGGCTGCTGCCAGATAGCTGACTCCACATTGTGATGTCG
GCACAGAGTTTGTCCGGTGAGGAATACGTGTGGAGATGGGTGAGGTGGTACTGGGCA
CTGGTGGGATTTTCCAAACTGTGGAGCAGGCAAGATTTTAGCCGCTCGAATTGGGCCA
TGTCGGACAGAGAAGAGCTCTTGTGCTTCGCCACTGATAGGGATGCTCCAGACCTGC
ATTCCATCACTGTAGCCAATCATAATCAACAAAGGCGGTTCACTCCCAGTACTATGTAT
TTCATGAAATTCCAGATTTCTTGATGTATCATTTAAATCTGCATTTTCAAATCTGACCCA
GACTATTTTCTCCTTTTCTTCTGTTAGAGGTGTTCCACTGTAAGCCTGTGGCACAACAT
CCTGCAGAAAAGTCACAACACTTTCCATGTAGGACTGCTCTGTGACAGCCTGGGGGC
GAACCACAACTCCACCAGTACAACGACTGGGTCTTCTTGGGGAATCTGTAGCCATAGC
TTCATTCATAAAACCGGCCGCCCCGCCGTTAACTTTCATCAAAGCCAGCAAACGCAGT
GTTCGGATCCGCGA3′
SEQ ID NO: 20: Sequence of MTAP / novel gene of SEQ ID NO: 2 fusion.
MTAP sequence (underlined) fused to novel gene of SEQ ID NO: 2:
5′TCATGCCTTCAAAGGTCAACTACCAGGCGAACATCTGGGCTTTGAAGGAAGA
GGGCTGTACACATGTCATAGTGACCACAGCTTGTGGCTCCTTGAGGGAGGAGATTCA
GCCCGGCGATATTGTCATTATTGATCAGTTCATTGACAGCTATGTCTCACAGTCCAGAC
TTGGAGTACAAGTAATAAGAAGAATAAAACTTGATCCCTTAAGTAGATTCACCATAAGT
TAGCTCAGAGCAATTCCAGTGCAAGTATGGTCTGTGATCCAGTAGTATCTTACAGACA
GCAAGTTGAACATTGTGGGATGCATGAGCTATTGAGGCCTTTGCAGCTTTCTGCTACA
TGGAGGCTAGGGCCAGAGTCAAGATTTATGCTTTGCAGCACACTGGTCAGCTGTTTTT
GCAAATCAGATTAAATGATTTTTAAATGAGGCTGAGAGCATGGGAGATACTAATGTGTG
TTTCCTTGTGAGCTACTGCATAAGTTAGGAAATTGAAATACAGAAAGATGAAAAGTGAT
TTGCCCAAGCATATAGATCAAAGCTGTGGCAGAACCAGGACTGGAACCTATATCTCTC
TACTAATGGTTTTTTTAAAAAAATAACCTTGTTTCAAAAATATTAAAAAGTCACAAGAAA
GGTAAACATGTGGATAAACAAAATGAAGAAAATAAAAATTATCCAGTAAAAAAAAAAAA
ACCTATAGTGAGTCGTATTAATTCGGATCCGC3′

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

In the invention the authors have identified molecular biomarker for cancer, in particular breast cancer, using entirely a new approach based on high-resolution oligonucleotide based array, the comparative genomic hybridization (a-CGH) (Agilent technologies). CGH is a technique in which differentially labeled tumor (or test) and reference DNA are hybridized to normal human metaphase chromosomes, followed by the analysis of the differences in fluorescence intensities of test and reference DNA along the entire length of chromosomes to identify regions of gains, deletions and amplifications. High-density oligo based a-CGH does not require direct chromosome analysis, construction of genomic or cDNA library. Based on this approach the inventors have isolated and characterized seven novel fusion genes involving 11 genes (Table 1).

TABLE 1
List of fusion genes cloned from the validation of CNT regions.

| Fusion gene | Genomic aberration |
|---|---|
| RCC2/CENPF | AMPLIFICATION/TRANSLOCATION |
| ARFGEF2/SULF2 | AMPLIFICATION/INVERSION |
| MTAP/New gene (SEQ ID | DELETION/IN FRAME FUSION |
| NO: 2) | |
| ATXN7/New gene (SEQ ID | AMPLIFICATION/TRANSLOCATION |
| NO: 1) | |
| BCAS3/ATXN7 | AMPLIFICATION/TRANSLOCATION |
| RPS6KB1/TMEM49 | AMPLIFICATION/INSERTION |
| RPS6KB1/EAP30 | AMPLIFICATION/INVERSION |

The a-CGH technique identified many Copy Number Transition (CNT) regions within known genes and in intergenic regions at a genomic interval from 2.7 kb to 23 kb and 2.7 kb to 4-75 kb respectively. Integrated molecular analysis by cytogenetics and molecular biology methods, including spectral karyotyping (SKY), FISH and RACE-PCR, and cloning approach were used to validate 48 of 83 CNT loci affecting known genes in MCF7. This study is the first of its kind to isolate fusion genes based only on the analysis of unbalanced copy number changes resolved at an unprecedented resolution.

Among the different commercially available oligo based CGH arrays, 244K array (Agilent Technologies) were selected in this study due to its unique array design providing an average resolution of about 6.4 kb and 16.5 kb in gene and intergenic regions respectively. Given the gene centric nature of 244K array all the CNT regions within 2.7 kb to 23 kb in known genes and 4 kb to 75 kb in intergenic regions could be identified (Table 2).

TABLE 2
List of Copy Number Transition Regions in MCF7

| | Chr | | | | | Strand | Gain/Loss | |
|---|---|---|---|---|---|---|---|---|
| GENE | No. | Band | CNT Start 5′ | CNT Stop 3′ | Size | (+/−) | 5′-3′ | |
| BX648145 | 1 | p22.3 | 85739643 | 85753011 | 13368 | (−) | L | L |
| NTNG1 | 1 | p13.3 | 107633996 | 107650115 | 16119 | (+) | G | N |
| BC017836 | 1 | p13.3 | 109933846 | 109944351 | 10505 | (+) | N | G |
| BC017836 | 1 | p13.3 | 109952006 | 109968401 | 16395 | (+) | G | N |
| KCND3 | 1 | p13.2 | 112069121 | 112078701 | 9580 | (−) | G | L |
| MAGI3 | 1 | p13.2 | 113749373 | 113757998 | 8625 | (+) | L | G |
| RSBN1 | 1 | p13.2 | 114050749 | 114060428 | 9679 | (−) | G | G |
| PHGDH | 1 | p12 | 119972493 | 119982982 | 10489 | (+) | L | G |
| LCE3D | 1 | q21.3 | 149365944 | 149369522 | 3578 | (−) | N | L |
| DUSP27 | 1 | q24.1 | 163819902 | 163832659 | 12757 | (+) | G | L |
| RASAL2 | 1 | q25.2 | 174797044 | 174802707 | 5663 | (+) | G | L |
| CACNA1E | 1 | q25.3 | 178397332 | 178406819 | 9487 | (+) | G | L |
| C1ORF120 | 1 | q25.3 | 179105887 | 179112799 | 6912 | (+) | L | G |
| NAV1 | 1 | q32.1 | 198463830 | 198475159 | 11329 | (+) | G | L |
| AK129946 | 1 | q32.1 | 198717889 | 198723672 | 5783 | (+) | L | G |
| CENPF | 1 | q41 | 211190840 | 211201667 | 10827 | (+) | L | G |
| PTPRG | 3 | p14.2 | 61579369 | 61586548 | 7179 | (+) | L | G |
| ATXN7 | 3 | p14.1 | 63901813 | 63916507 | 14694 | (+) | G | N |
| ATXN7 | 3 | p14.1 | 63948876 | 63955584 | 6708 | (+) | N | G |
| AK057923 | 3 | p14.1 | 64917886 | 64937725 | 19839 | (+) | G | L |
| PPM1L | 3 | q26.1 | 162226371 | 162232595 | 6224 | (+) | N | G |
| MGC48628 | 4 | q22.1 | 91848619 | 91856061 | 7442 | (+) | L | N |
| AB040888 | 4 | q35.1 | 183994785 | 184013382 | 18597 | (+) | L | L |
| AB095936 | 6 | q25.2-25.3 | 155425418 | 155440143 | 14725 | (+) | N | L |
| LOC223075 | 7 | p15.1 | 31416106 | 31427086 | 10980 | (+) | N | G |
| TBX20 | 7 | p14.3 | 35050350 | 35061441 | 11091 | (−) | L | G |
| AUTS2 | 7 | q11.22 | 69437105 | 69447117 | 10012 | (+) | N | L |
| AUTS2 | 7 | q11.22 | 69702445 | 69709454 | 7009 | (+) | L | N |
| AJ007770 | 7 | q32 | 141494752 | 141511878 | 17126 | (+) | N | L |
| AL007770 | 7 | q34 | 141518287 | 141527039 | 8752 | (+) | L | N |
| FAM62B | 7 | q36.3 | 158091231 | 158098626 | 7395 | (−) | N | G |
| RNF170 | 8 | p11.21 | 42849186 | 42866053 | 16867 | (−) | L | L |
| CA1 | 8 | q21.2 | 86464908 | 86478202 | 13294 | (−) | N | G |
| MTAP | 9 | p21.3 | 21822787 | 21827873 | 5086 | (+) | L | L |
| BC063022 | 11 | q14.2 | 86233396 | 86255081 | 21685 | (+) | N | L |
| RNF214 | 11 | q23.3 | 116629867 | 116641228 | 11361 | (+) | L | N |
| AK097820 | 11 | q23.3 | 118864986 | 118880819 | 15833 | (+) | N | L |
| SLC2A13 | 12 | q12 | 38607250 | 38614376 | 7126 | (−) | N | L |
| SLC2A13 | 12 | q12 | 38693079 | 38705855 | 12776 | (−) | L | N |
| BC041395 | 13 | q21.2 | 59192999 | 59212192 | 19193 | (−) | N | L |
| MGC48595 | 14 | q24.3 | 73060360 | 73071404 | 11044 | (−) | G | L |
| MGC48595 | 14 | q24.3 | 73075559 | 73082250 | 6691 | (−) | L | N |
| GM88 | 15 | q14 | 32511534 | 32517513 | 5979 | (−) | N | L |
| GM88 | 15 | q14 | 32605144 | 32628679 | 23535 | (−) | L | L |
| C15ORF33 | 15 | q21.1 | 47521758 | 47532595 | 10837 | (−) | L | G |
| FGF7 | 15 | q21.1 | 47521758 | 47532595 | 10837 | (+) | L | G |
| UNC13C | 15 | q21.3 | 52344881 | 52350058 | 5177 | (+) | G | L |
| BC036541 | 15 | q21.3 | 54823498 | 54832642 | 9144 | (−) | N | L |
| TLN2 | 15 | q22.2 | 60814580 | 60819580 | 5000 | (+) | L | G |
| LIA10 | 16 | q22.1 | 65717456 | 65726391 | 8935 | (+) | L | G |
| UAC14 | 16 | q22.1 | 69298360 | 69308884 | 10524 | (−) | N | G |
| USP6 | 17 | p13.2 | 4981551 | 4988992 | 7441 | (+) | L | G |
| AK125954 | 17 | p11.2 | 20551151 | 20569693 | 18542 | (+) | N | G |
| AK125954 | 17 | p11.2 | 20582325 | 20589698 | 7373 | (+) | L | G |
| SSH2 | 17 | q11.2 | 25231047 | 25242511 | 11464 | (−) | L | N |
| BC006271 | 17 | q21.31-q21.32 | 42312705 | 42324184 | 11479 | (−) | L | G |
| TOB1 | 17 | q21.33 | 46296315 | 46303822 | 7507 | (−) | G | N |
| TEX14 | 17 | q22 | 53989180 | 53997246 | 8066 | (−) | A | A |
| FAM33A | 17 | q22 | 54551774 | 54558434 | 6660 | (−) | A | A |
| TMEM49 | 17 | q23.1 | 55260272 | 55262899 | 2627 | (+) | A | A |
| BCAS3 | 17 | q23.2 | 56222422 | 36240772 | 18350 | (+) | A | A |
| BCAS3 | 17 | q23.2 | 56631581 | 56645691 | 14110 | (+) | A | A |
| INTS2 | 17 | q23.2 | 57336887 | 57344164 | 7277 | (−) | A | A |
| PECAM1 | 17 | q23.3 | 59767616 | 59781504 | 13888 | (−) | A | A |
| SLC25A19 | 17 | q25.1 | 70785590 | 70793471 | 7881 | (−) | N | G |
| ZC3HDC5 | 17 | q25.1 | 71323386 | 71332309 | 8923 | (+) | G | N |
| MYOM1 | 18 | p11.31 | 3197218 | 3205247 | 8029 | (−) | N | L |
| OLFM2 | 19 | p13.2 | 9899170 | 9906235 | 7065 | (−) | N | L |
| MYO9B | 19 | p13.11 | 17077587 | 17088803 | 11216 | (+) | G | L |
| FCHO1 | 19 | q13.11 | 17720415 | 17732926 | 12511 | (+) | L | N |
| BC063593 | 20 | p13 | 3803666 | 3810027 | 6361 | (+) | G | L |
| PTPRT | 20 | q12-q13.11 | 40728551 | 40736791 | 8240 | (−) | N | G |
| EYA2 | 20 | q13.12 | 45198347 | 45205141 | 6794 | (+) | G | A |
| EYA2 | 20 | q13.12 | 45205194 | 45214159 | 8965 | (+) | A | G |
| EYA2 | 20 | q13.12 | 45222780 | 45232779 | 9999 | (+) | G | A |
| ARFGEF2 | 20 | q13.13 | 46972419 | 46978778 | 6359 | (+) | A | G |
| SLC9A8 | 20 | q13.13 | 47913043 | 47921764 | 8721 | (+) | G | G |
| BCAS4 | 20 | q13.13 | 48854956 | 48869571 | 14615 | (+) | G | G |
| AK024093/ZNF217 | 20 | q13.2 | 51611532 | 51618614 | 7082 | (+/−) | G | A |
| ZNF217 | 20 | q13.2 | 51611532 | 51618614 | 7082 | (−) | G | A |
| BC047656 | 20 | q13.31 | 55279595 | 55296265 | 16670 | (+) | G | A |
| IL1RAPL1 | X | p21.3-p21.2 | 29083972 | 29096164 | 12192 | (+) | N | L |
| IL1RAPL1 | X | p21.3-p21.2 | 29158574 | 29167881 | 9307 | (+) | L | N |
| Unknown | 1 | p21.1 | 106433497 | 106457644 | 24147 | | L | G |
| Unknown | 1 | p13.3 | 112265246 | 112298230 | 32984 | | G | N |
| Unknown | 1 | p13.1 | 115068654 | 115092893 | 24239 | | G | G |
| Unknown | 1 | q21.3 | 149247196 | 149278545 | 31349 | | G | N |
| Unknown | 1 | q21.3 | 149399354 | 149403519 | 4165 | | L | N |
| Unknown | 1 | q23.1 | 154890767 | 154903613 | 12846 | | L | G |
| Unknown | 1 | q23.1 | 155218065 | 155227871 | 9806 | | G | L |
| Unknown | 1 | q24.1 | 163264221 | 163274862 | 10641 | | L | G |
| Unknown | 1 | q24.2 | 165065971 | 165085938 | 19967 | | L | G |
| Unknown | 1 | q25.2 | 175640962 | 175648727 | 7765 | | L | G |
| Unknown | 1 | q32.2 | 204298490 | 204311956 | 13466 | | G | L |
| Unknown | 1 | q41 | 216151589 | 216175292 | 23703 | | G | N |
| Unknown | 3 | p22.1 | 41184892 | 41202721 | 17829 | | N | L |
| Unknown | 3 | q13.31 | 117652389 | 117674767 | 22378 | | N | L |
| Unknown | 3 | q13.31 | 118314647 | 118357700 | 43053 | | L | N |
| Unknown | 4 | q34.3 | 181892216 | 181911919 | 19703 | | N | L |
| Unknown | 4 | q34.3 | 182147786 | 182173613 | 25827 | | L | L |
| Unknown | 4 | q34.3 | 182713649 | 182752068 | 38419 | | L | L |
| Unknown | 4 | q34.3 | 183192305 | 183222741 | 30436 | | L | L |
| Unknown | 6 | q14.1 | 78983335 | 79035891 | 52556 | | N | L |
| Unknown | 6 | q14.1 | 79080047 | 79101978 | 21931 | | L | N |
| Unknown | 8 | q24.21 | 129972316 | 129988070 | 15754 | | G | G |
| Unknown | 9 | p21.3 | 22060042 | 22076798 | 16756 | | L | N |
| Unknown | 11 | p11.21 | 45773986 | 45782630 | 8644 | | L | G |
| Unknown | 11 | q12.1 | 59202839 | 59210980 | 8141 | | L | N |
| Unknown | 12 | p13.31 | 9452847 | 9528590 | 75743 | | N | L |
| Unknown | 12 | p13.31 | 9585215 | 9613074 | 27859 | | L | N |
| Unknown | 12 | p13.2 | 11393532 | 11404653 | 11121 | | N | L |
| Unknown | 12 | p13.2 | 11430946 | 11444721 | 13775 | | L | N |
| Unknown | 13 | q14.13 | 45989799 | 46006899 | 17100 | | N | G |
| Unknown | 13 | q14.2 | 46994706 | 47023674 | 28968 | | G | L |
| Unknown | 15 | q11.2 | 22021233 | 22038880 | 17647 | | N | L |
| Unknown | 15 | q11.2 | 22055612 | 22077517 | 21905 | | L | N |
| Unknown | 20 | p12.3 | 6713052 | 6717170 | 4118 | | L | G |
| Unknown | 20 | q12.3 | 33362926 | 33386939 | 24013 | | N | L |
| Unknown | 20 | q22.13 | 38288415 | 38310473 | 22058 | | L | G |
| Unknown | 20 | q12 | 38927294 | 38953992 | 26698 | | N | G |
| Unknown | 20 | q13.13 | 48709672 | 48723763 | 14091 | | G | A |
| Unknown | 20 | q13.2 | 50034067 | 50074064 | 39997 | | G | G |
| Unknown | 20 | q13.2 | 52938182 | 52993159 | 54977 | | A | G |
| Unknown | 20 | q13.31 | 55104257 | 55111937 | 7680 | | G | A |

The present invention therefore provides the use of CGH technique for the identification of CNT regions comprising fused genes. All the fusion genes identified in this study were the product of genomic perturbations in genes at copy number transition (CNT) regions, or boundaries of amplifications and deletions, detected in the size range from 30 kb to 1 mb, a resolution not achievable by chromosome based and other CGH methods. Detailed analysis of CNT regions using 244K array revealed the precise identification of rearrangements within known genes. Further characterization of CNT regions by FISH and RACE-PCR approach identified novel fusion transcripts listed in Table 1 above.

Accordingly, the present invention provides an isolated fused gene comprising at least one first gene and/or fragment thereof fused to at least one second gene and/or fragment thereof, wherein at least the first and/or the second gene, independently, is selected from the group of genes consisting of: RCC2, CENPF, ARFGEF2, SULF2, MTAP, ATXN7, BCAS3, RPS6 KB1, TMEM49, EAP30, a gene having the nucleotide sequence SEQ ID NO:1, and a gene having the nucleic acid SEQ ID NO:2, or a fragment thereof. The first and the second gene, independently, may be selected from the group consisting of: RCC2, CENPF, ARFGEF2, SULF2, MTAP, ATXN7, BCAS3, RPS6 KB1, TMEM49, EAP30, a gene having the nucleotide sequence SEQ ID NO:1, and a gene having the nucleic acid SEQ ID NO:2, or a fragment thereof. Accordingly to a particular aspect of the invention, the first gene and the second gene may have inverted position within the fused gene. According to a particular aspect, the first gene may be selected from the group consisting of: RCC2, ARFGEF2, MTAP, ATXN7, BCAS3, and RPS6 KB1, or a fragment thereof. According to a particular aspect, the second gene may be selected from the group consisting of: CENPF, SULF2, a gene having the nucleotide sequence SEQ ID NO:1, a gene having the nucleotide sequence of SEQ ID NO:2, ATXN7, TMEM49, and EAP30, or a fragment thereof. According to one or more embodiment, the first and/or the second gene is ATXN7. According to another embodiment, the first and/or the second gene is ARFGEF2. According to another embodiment, the first and/or the second gene is SULF2. The first and/or second gene may be RPS6 KB1. According to another embodiment, the first and/or second gene is a gene comprising the nucleotide sequence SEQ ID NO:1 or SEQ ID NO:2 or a fragment thereof. The fusion of the genes may be by genomic translocation, insertion, inversion, amplification and/or deletion.

A “fusion gene” as used herein refers to a hybrid gene formed from two previously separate genes and thus resulting in gene rearrangement. Alternatively, the separate genes may undergo rearrangement independently before they fuse to each other. Accordingly “fused gene” may be construed accordingly to refer to any such rearrangement event. Fused genes can occur as the result of mutations such as translocation, deletion, inversion, amplification and/or insertion.

“Translocation” of genes results in a chromosome abnormality caused by rearrangement of parts between nonhomologous chromosomes. It is detected on cytogenetics or a karyotype of affected cells. “Deletions” in chromosomes may by of the entire gene or only a portion of the gene. Genetic “insertion” is the addition of one or more nucleotide base pairs into a genetic sequence. This can often happen in microsatellite regions due to the DNA polymerase slipping. An “inversion” is rearrangement of genes in a chromosome in which a segment of a gene is reversed end to end. An “amplification” results when a DNA is amplified resulting in the gain in copy number.

The fused gene may be selected from the group of fused genes RCC2/CENPF, ARFGEF2/SULF2, MTAP/a gene comprising the nucleotide sequence SEQ ID NO:2, ATXN7/a gene comprising the nucleotide sequence SEQ ID NO:1, BCAS3/ATXN7, RPS6 KB1/TMEM49, and RPS6 KB1/EAP30, or fragment(s) thereof. In particular, the fused gene may be ARFGEF2/SULF2 fusion gene comprising the nucleic acid sequence of SEQ ID NO: 16 and/or a fragment thereof. More in particular the fused gene may be RPS6 KB1/TMEM49 fusion gene comprising the nucleic acid sequence of SEQ ID NO: 17 and/or a fragment thereof. The fused gene may further be ATXN7/a gene having the nucleotide sequence SEQ ID NO:1 gene fusion comprising the nucleic acid sequence of SEQ ID NO: 18 and/or a fragment thereof. The fused gene may be ATXN7/BCAS3 fusion gene comprising the nucleic acid sequence of SEQ ID NO: 19 and/or a fragment thereof. The fused gene may also be MTAP /a gene having the nucleotide sequence SEQ ID NO:2, the gene fusion comprising the nucleic acid sequence of SEQ ID NO: 20 and/or a fragment thereof.

The fused genes are written together in the form of gene“x”/gene“y”. Therefore the fused genes are referred to in this, form throughout this application.

The fused genes may be in any suitable vector, phage, plasmid, or a fragment comprising the fused gene. There is no limit in the size of the nucleic acid construct and the fused gene.

There is also provided an isolated nucleic acid molecule comprising the nucleotide sequence SEQ ID NO:1 and/or SEQ ID NO:2, or a fragment thereof. The isolated nucleic acid may be comprised in a vector. The vector may be any suitable vector, phage, plasmid, or nucleic acid fragment comprising the nucleic acid molecule of SEQ ID NO: 1 and/or SEQ ID NO: 2. There is no limit in the size of the nucleic acid construct and the nucleic acid molecule.

According to another aspect the invention provides a diagnostic and/or prognostic kit for the diagnosis and/or prognosis of tumour in a subject comprising detecting at least one fused gene, wherein the presence of the fused gene is indicative of presence and/or the stage of tumour.

There is also provided a diagnostic and/or prognostic kit, wherein the kit comprises at least one nucleic acid molecule capable of hybridizing to and/or complementary to the fused gene and/or a fragment thereof, wherein hybridization is indicative of presence and/or the stage of tumour.

The present invention further provides a diagnostic and/or prognostic kit for the diagnosis and/or prognosis of tumour in a subject, wherein the kit comprises one or more fragment representative of a genome capable of hybridizing to differentially labelled genomic DNA isolated from tumour tissue from at least one subject and from a control tissue, wherein an increase or decrease of the hybridization intensity and/or signal(s) of the label in the tumour tissue, compared to that in control tissue detects copy number transition (CNT) regions in the tumour tissue, indicative of presence and/or stage of tumour.

The CNT regions may comprise fused gene(s).

“Diagnose” or “diagnosis” used herein, refers to determining the nature or the identity of a condition (disease). A diagnosis may be accompanied by a determination as to the severity of the disease. “Prognostic” or “prognosis” used herein refers to predicting the outcome or prognosis of a disease, such as to give a chance of survival based on observations and results of clinical tests. “Predisposition” used herein refers to the likelihood of being diagnosed with, or susceptibility to a particular disease.

“Copy number transitions (CNT) regions” refer to boundaries of genomic perturbations due to deletions, insertions, inversions, amplifications described previously in earlier section, that result in the variation the copy number of the genes present therein. The current invention is the first study wherein the fusion genes were isolated based on the analysis of these copy number changes. The invention used the CGH technique to identify CNT regions within known genes. “CGH or Comparative genome hybridization” method used herein analysed copy number changes (gains/losses) in the DNA content. The method is well known to those skilled in the art. CGH is capable of detecting loss, gain and amplification of the copy number at the levels of chromosomes. The use of array CGH overcomes many of these limitations, with improvement in resolution and dynamic range, in addition to direct mapping of aberrations to the genome sequence and improved throughput. The DNA may be isolated from a tumor tissue and from control tissue by standard methods known in the art. The labeling of the DNA is also well known in the art. The fused genes comprised in the CNT regions may be detected by FISH and/or RACE technique. Fused gene may be any one of the fused gene described in the earlier sections.

The term “nucleic acid” is well known in the art and is used to generally refer to a molecule (one or more strands) of DNA, RNA or a derivative or analog thereof comprising nucleobases. A nucleobase includes, for example, a purine or pyrimidine base found in DNA (e.g., an adenine “A”, a guanine “G”, a thymine “T” or a cytosine “C”) or RNA (e.g., an A, a G, an Uracil “U” or a C). The term nucleic acid encompasses the terms “oligonucleotide” and “polynucleotide” each as subgenus of the term “nucleic acid”. The term “complementary” in the context of nucleic acids refers to a strand of nucleic acid non-covalently attached to another strand, wherein the complementarity of the two strands is defined by the complementarity of the bases. For example, the base A on one strand pairs with the base T or U on the other, and the base G on one strand pairs with the base C on the other. An oligonucleotide or analog is of “substantial complementarity” when there is a sufficient degree of complementarity to avoid non-specific binding of the oligonucleotide or analog to non-target sequences under conditions in which specific binding is desired

A nucleic acid molecule is “hybridisable” to another nucleic acid molecule (in the present case, the miR183), when a single-stranded form of the nucleic acid molecule can anneal to the other nucleic acid molecule under the appropriate conditions of temperature and solution ionic strength (Sambrook and Russell, 2001). The conditions of temperature and ionic strength determine the “stringency” of the hybridisation. Hybridisation requires the two nucleic acids to contain complementary sequences. Depending on the stringency of the hybridisation, mismatches between bases are possible. The appropriate stringency for hybridising nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridisation decreases in the following order: RNA:RNA, DNA:RNA, DNA:DNA. For hybrids of greater than 100 nucleotides in length, equations for calculating Tm have been derived (Sambrook and Russell, 2001). For hybridisation with shorter nucleic acids, i.e. oligonucleotides, the position of mismatches becomes more important, and the length of the oligonucleotide determines its specificity (Sambrook and Russell, 2001).

The DNA may be isolated from a tumour tissue. The tumour is stage III tumour, wherein the tumour is solid tumour. In particular the tumour may be breast tumour. The tumour tissue may be from a subject suffering from the tumour.

A “subject” may be a patient suffering from the tumour, in particular solid tumour, for example, breast tumour. A person skilled in the art will know how to select subjects based on their amenability to a particular treatment, or their susceptibility to a particular disease.

The “control” for example, may not be suffering from tumour. The control may exhibit control level label intensity and/or signal from the labelled DNA. The “control value” may also be an average value in expression obtained from a selected population.

The stage of a tumour is a descriptor (usually numbers I to IV) of how much the cancer has spread. The stage often takes into account the size of a tumor, how deep it has penetrated, whether it has invaded adjacent organs, if and how many lymph nodes it has metastasized to, and whether it has spread to distant organs. Staging of cancer is important because the stage at diagnosis is the most powerful predictor of survival, and treatments are often changed based on the stage. Correct staging is critical because treatment is directly related to disease stage. Thus, incorrect staging would lead to improper treatment, and material diminution of patient survivability. Correct staging, however, can be difficult to achieve. Staging systems are specific for each type of cancer (e.g. breast cancer).

Overall Stage Grouping is also referred to as Roman Numeral Staging. This system uses numerals I, II, III, and IV (plus the 0) to describe the progression of cancer. Stage 0 cancers are carcinoma in situ. Stage I cancers are localized to one part of the body. Stage II cancers are locally advanced, as are Stage III cancers. Whether a cancer is designated as Stage II or Stage III can depend on the specific type of cancer; for example, in Hodgkin's disease, Stage H indicates affected lymph nodes on only one side of the diaphragm, whereas Stage III indicates affected lymph nodes above and below the diaphragm. The specific criteria for Stages II and III therefore differ according to diagnosis. Stage IV cancers have often metastasized or spread to other organs or throughout the body.

According to yet another aspect, the invention provides a method of diagnosis and/or prognosis of presence and/or stage of tumour in a subject comprising detecting at least one fused gene, wherein the presence of the fused gene is indicative of presence and/or the stage of tumour.

The method may comprise providing at least one nucleic acid molecule capable of hybridizing to and/or complementary to the fused gene and/or a fragment thereof, wherein hybridization is indicative of presence and/or the stage of tumour.

According to a further aspect there is provided a method of diagnosis and/or prognosis of presence and/or stage of tumour in a subject, wherein the method comprises providing one or more fragments representative of a genome capable of hybridizing to differentially labelled genomic DNA isolated from tumour tissue from at least one subject and from a control tissue, wherein an increase or decrease of the hybridization intensity and/or signal(s) of the label in the tumour tissue, compared to that in control tissue detects copy number transition (CNT) regions in the tumour tissue, indicative of presence and/or stage of tumour. In particular, the CNT regions comprise fused gene(s). The fused genes may be detected by FISH and/or RACE technique.

The method of diagnosis and/or prognosis may be for stage III tumour, in particular solid tumours. In particular, the tumour may be breast tumour.

There is further provided a kit for the detecting the presence of fused genes, wherein the kit comprises one or more fragments representative of a genome capable of hybridizing to differentially labelled control and test genomic DNA wherein an increase or decrease of the hybridization intensity and/or signal(s) of test genome, compared to that in control genome detects copy number transition (CNT) regions in the test genome, wherein the CNT regions comprise fused genes.

According to yet another aspect, the invention provides a method of detecting the presence of fused genes, wherein the method comprises providing one or more fragments representative of a genome capable of hybridizing to differentially labelled control and test genomic DNA wherein an increase or decrease of the hybridization intensity and/or signal(s) of test genome, compared to that in control genome detects copy number transition (CNT) regions in the test genome, wherein the CNT regions comprise fused genes.

The “test genomic DNA” as used herein refers to the labelled genomic DNA to be compared with a control DNA. The test genomic DNA is understood to have the same meaning as DNA isolated from a tumour tissue of a subject.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

Array comparative Genomic Hybridization (a-CGH)

Oligo nucleotide based array comparative genomic hybridization is an emerging technology designed for high precision mapping of unbalanced copy number changes (Barrett et al., 2004). Poor resolution limits in metaphase chromosome based CGH, cDNA array CGH and BAC clone array CGH detected copy number change boundaries within a large genomic distance of more than 100 kb to several megabases. The SNP array with high density probes from Affymetrix can be used for copy number analysis, but the probes are mostly selected from intergenic regions and further validation studies are required to map breakpoints within genes. In this study the recently introduced version (244K array) of the oligo CGH array from Agilent Technologies, USA, which contains 244,000 probes providing a genome wide average resolution of ˜6.4 kb to 16.5 kb and even higher resolution within in genes (<3-10 kb) was used. Array features include mainly probes from the well known and cancer related genes and a minimal number of probes are derived from intergenic regions. Given the unique design and reproducibility of this method high precision mapping of genomic rearrangements and copy number changes are obtained with remarkable specificity. Although this method is developed and available through commercial sources, it allows us to custom design the array by selecting probes at even higher density for a genomic region of interest which allow us to design our own array to achieve resolution in the range of less than 1 kb for a given region.

### Identification of Copy Number Transition Region (CNT)

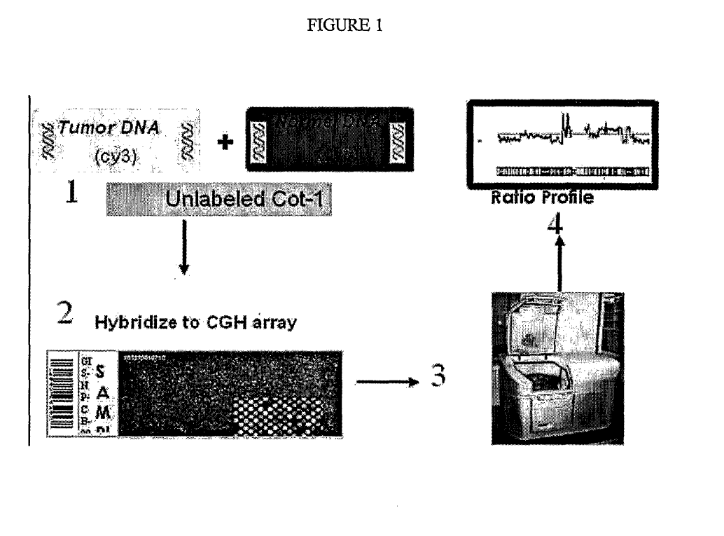

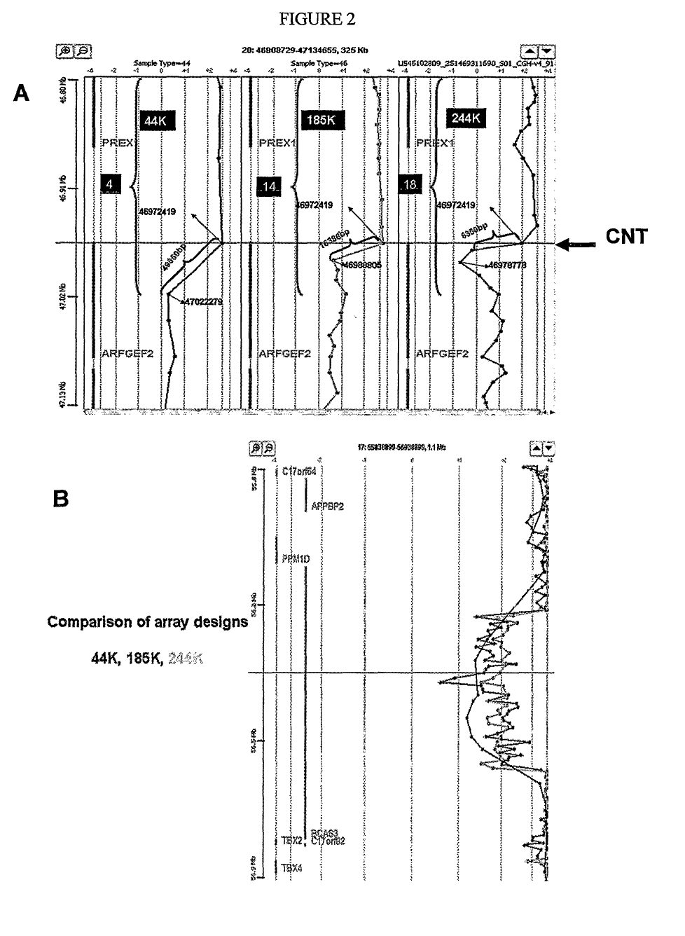

Oligonucleotide comparative genomic hybridization is a high-resolution method to detect unbalanced copy number changes at whole genome level. Competitive hybridization of differentially labelled tumor and reference DNA to oligonucleotide printed in an array format (Agilent Technologies, USA) and analysis of fluorescent intensity for each probe will detect the copy number changes in the tumor sample relative to normal reference genome (FIG. 1). Using this method, the present inventors identified whole chromosome gains, losses, and more importantly many regions of gains and losses at sub microscopic level in the size range of <30 kb. Initially, three different array designs (43K, 185K and 244K) of oligo array for MCF7 were tested. The 244K array provided an average resolution, of 6.5 kb and 16.5 in gene and intergenic regions, thus allowing mapping the copy number transition (CNT) regions at an unprecedented resolution. The CNT regions based on copy number transition including at least two or more probes in the flanking regions for loss or gain of at least one copy were selected. Comparison of different array design for a CNT region in ARFGEF2 gene was detected within 49.8 kb, 16.3 kb and 6.3 kb in 44K, 185K and 244K arrays respectively (FIG. 2).

### High Resolution Method to Detect Unbalanced Chromosomal Changes

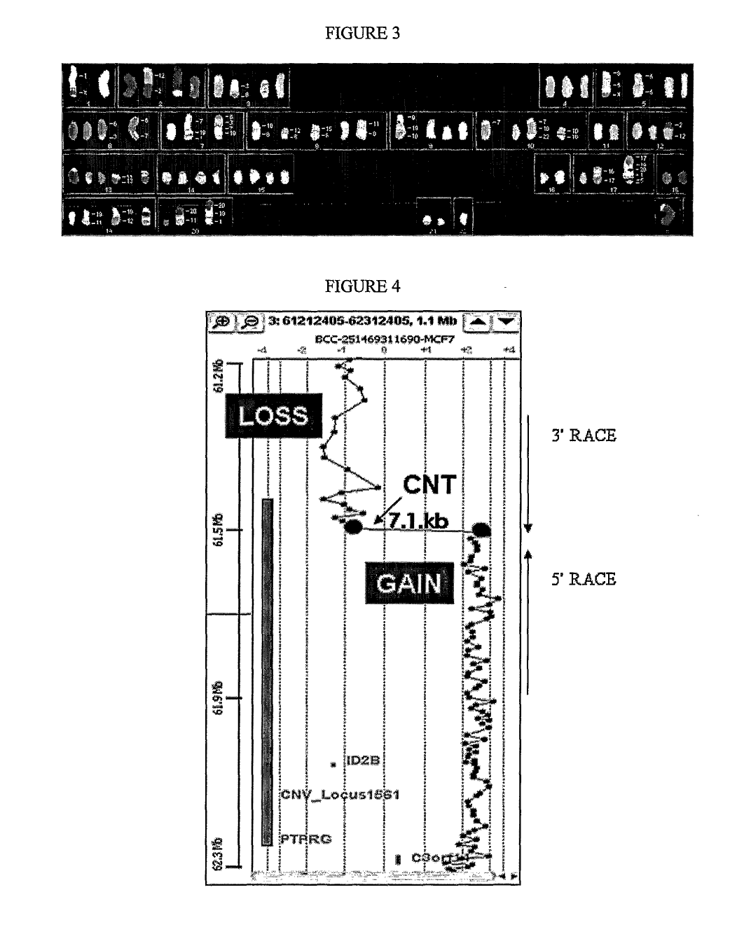

Based on the best resolution detected in 244 K array MCF7 cell lines known to contain many unbalanced structural and numerical aberrations were analyzed (FIG. 3).

Strategy to Isolate Fusion Gene from a CNT Region (FIG. 4)

Select CNT region within a gene

Confirm genomic rearrangement by fluorescence in situ hybridization

Identify genomic interval of CNT region

Design primer from the region present in at least one copy

Avoid regions that are involved in homozygous deletion

Design primers from exons close to the CNT region

Decide on 5′ or 3′ RACE depending on the orientation of the gene

Clone PCR product and sequence

Confirm RACE PCR results by RT PCR using a primer from the known and the new gene

Using the strategy described above, the present inventors validated 48 genes containing CNT regions in MCF7 cell line and isolated seven novel fusion genes described in the following sections.

Gene 1: RCC2/CENPF (SEQ ID NO: 15) rearranged at 1(q41)

### Isolation of a Truncated Form of CENPF Gene Produced by Genomic Rearrangement

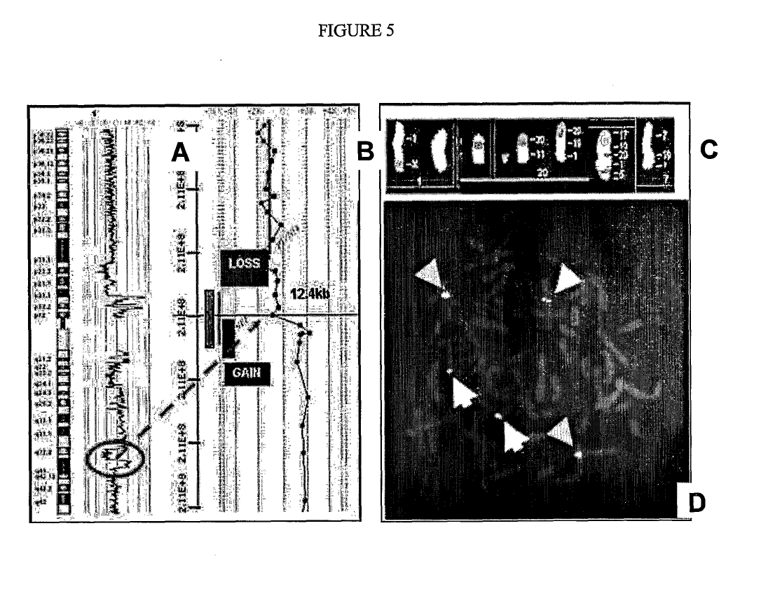

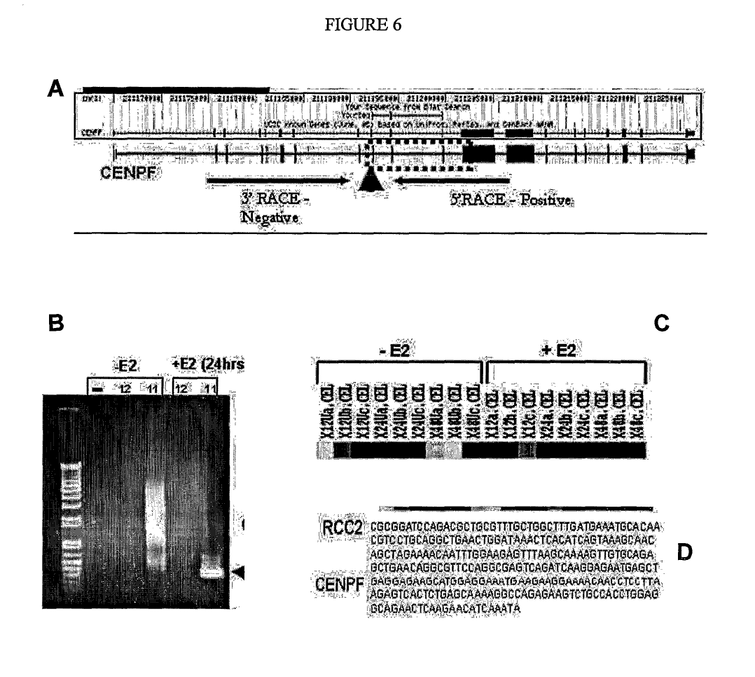

CNT region in CENPF gene with the genomic interval of 10,827 bp between 5′211190840 and 3′211201667 containing exons 9, 10 and 11 was identified. The 5′ end of the gene is present in at least one copy and 3′ region amplified to at least three copies. FISH analysis using BAC clones (RP11-281J12, 3′end and RP11-37015, 5′end) confirmed rearrangement of CENPF with at least three locations rather than tandem duplication on the same chromosome. Spectral karyotyping analysis revealed one copy of normal chromosome 1 and a second copy rearranged with chromosome X, in addition small segments of chromosome 1 inserted in at least five different locations (FIG. 5). Further to the confirmation of rearrangement by FISH analysis, primers were designed from exon 6 (5′ GTGTTCTCATGGCAGCAAGA 3′) (SEQ ID NO: 3) and 11 (CTGTTTGATGTTCTTGAGTTCTGC3′) (SEQ ID NO: 4) and 3′ and 5′RACE respectively was performed, using total RNA from MCF7 treated with estradiol (E2) and untreated cells. We selected RNA from E2 cells because, gene expression analysis showed expression of CENPF gene only at 24 hours after treatment with E2. PCR results were negative for 3′RACE confirming absence of normal CENPF transcript consistent with a-CGH data showing deletion of at least two copies at the 5′ end of the gene. 5′RACE PCR amplified a 270 bp product only in RNA from cells treated with E2 consistent with gene expression data (FIG. 6B). 5′RACE PCR results were confirmed by RT PCR using primers from RCC2 (5′ TGCGTTTGCTGGCTTTGAT3′) (SEQ ID NO: 5) and CENPF exon 11 5′ (CTGTTTGATGT TCTTGAGTTCTGC3′) (SEQ ID NO: 4).

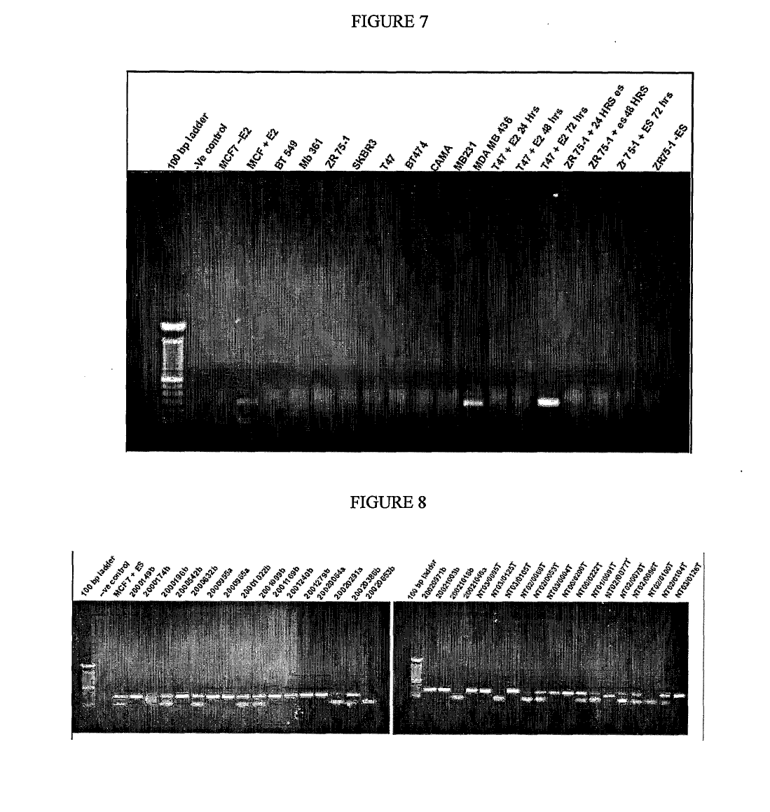

The PCR product was cloned into a plasmid vector using TA cloning kit (Invitrogen, USA) and sequence analysis showed the breakpoint in exon 9 and a 46 bp upstream sequence matching the 5′ end of RCC2 gene. Surprisingly, the 46 bp RCC2 sequence matched only to the mRNA sequence in the GENBANK by BLAST search, but not to the genomic sequence of RCC2. FISH validation for confirmation of fusion of RCC2 with CENPF was negative. Further analysis of sequence starting from the breakpoint in exon 9 of CENPF and the rest of the 3′ end sequence confirmed a perfect open reading frame (ORF) starting from the breakpoint immediately upstream of ATG sequence in exon 9. Although the 3′RACE PCR was negative in both RNA's we performed RT PCR using primers from exon 7 and 11 of CENPF and confirmed the absence of normal transcript which indicated the expression of only truncated form of CENPF. Further validation by RT PCR using RNA from cell lines and primary breast cancer tumors showed amplification in cell lines T47D (72 hours after E2 treatment), and MDAMB 436 under normal condition (FIG. 7) and in about 50% (17/35) of primary breast cancer tumors (FIG. 8). The inventors further evaluated the presence of normal CENPF transcript in all primary tumor samples using primers from exon 7 and 11 and found that only 12 out of 35 tumors were positive, indicating the expression of only truncated form of CENPF in majority of tumors. Further validation in additional tumors is in progress (FIG. 9).

These results provide evidence for the isolation of a rearranged gene from a CNT region without any direct evidence from conventional karyotyping. Further the results show that the expression of CENPF is regulated by E2 and the CENPF is expressed in a truncated form in majority of breast cancer tumors. These results also indicate the role of CENPF in centromere kinetocore assembly during cell division. Importantly the invention suggests that a high level expression of truncated CENPF is seen in grade 3 primary breast cancer tumors and the aberrant CENPF protein may be causative factor for abnormal segregation of chromosomes during mitosis leading to aneuploidy.

Isolation of Fusion Genes from the Commonly Amplified Regions in Breast Cancer: Characterization of Amplifications in Breast Cancer

The randomness of most of the chromosome rearrangements between different breast cancer tumors might not yield a specific recurrent chromosome aberration, however, it has been shown that 17q23 and 20q13 regions are recurrently amplified in 20-39% of primary breast cancer with distinct clinical outcome. An in depth characterization of these two amplicons revealed many CNT regions affect genes known to be over expressed in breast cancer but none of them were identified as fusion genes except BCAS4 and BCAS3 (Barlund et al., 2002). Three novel fusion genes were isolated using the CNT in the amplicons using the present inventors' new approach. In MCF7, throughout the genome there were many amplified regions from 3 copies to more than 40 copies, particularly at 17q23 and 20q13. The 17q23 amplification reported in 20% of primary breast tumors and many genes including RPS6 KB1, MUL, APPBP2, and TRAP240 are known to be over expressed. Similarly, genes AIB1, ZNF217, BTAK, and NABC1 in 20q13 amplification reported to be over expressed in 12-39% of primary breast tumor (Kallioniemi et al., 1994, Muleris, et al., 1994). High-level amplification of 20q13 may be an indicator of poor clinical outcome in node-negative breast cancer. The 17q23 amplicon revealed genes that may have oncogenic potential and may contribute to the more aggressive clinical course in breast cancer patients. All the genes in this amplicon showed variable level of expression and further variations in expression found in different probes for PRKCBP1 gene, indicating additional rearrangements within amplicons without showing an obvious CNT. Contrary to the conventional interpretation, these results indicate that amplicons are the rich source of rearrangements and the chance for identifying novel fusion genes are much higher in amplified regions. Further detailed analysis for all the genes within amplicons are described in detail in the in the following sections.

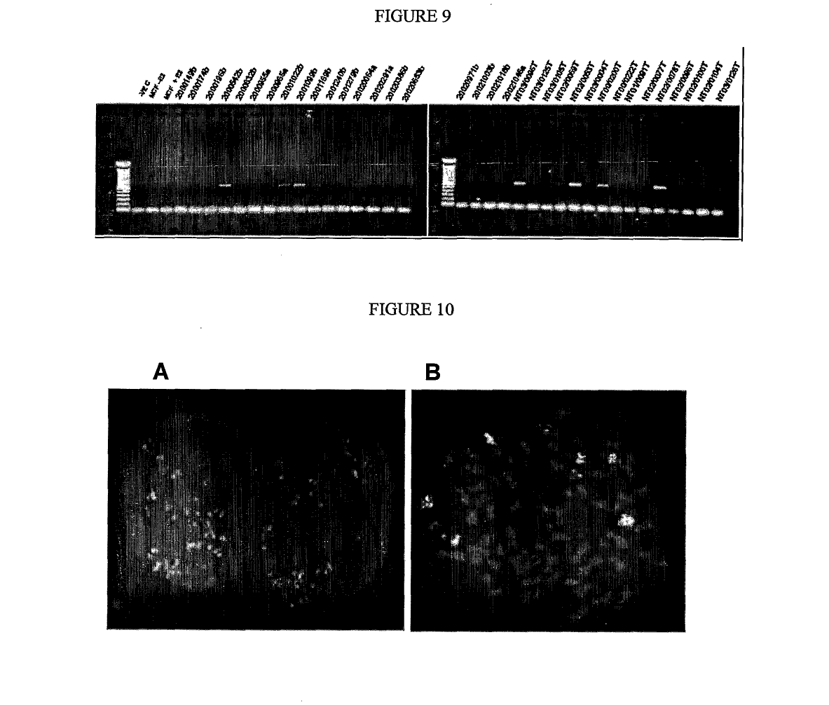

The present inventors further attempted to understand the genomic organization of the amplified regions in MCF7 for which we performed FISH analysis using a BAC clone for BRIP1 (RP11-482H10) gene within the amplified region at 17q23. FISH results indicated that the amplified sequences are inserted at many locations within the genome (FIG. 10) confirming the added complexity of the rearrangements. The uneven distributions of signal intensity of the amplified signals at different locations indicate further rearrangements. Such cryptic rearrangements are not detectable even with high-resolution array CGH.

Gene 2: ARFGEF2/SULF2 (SEQ ID NO: 16) inv(20q13.13)
Isolation of a Fusion Gene Produced by Inversion within an Amplicon

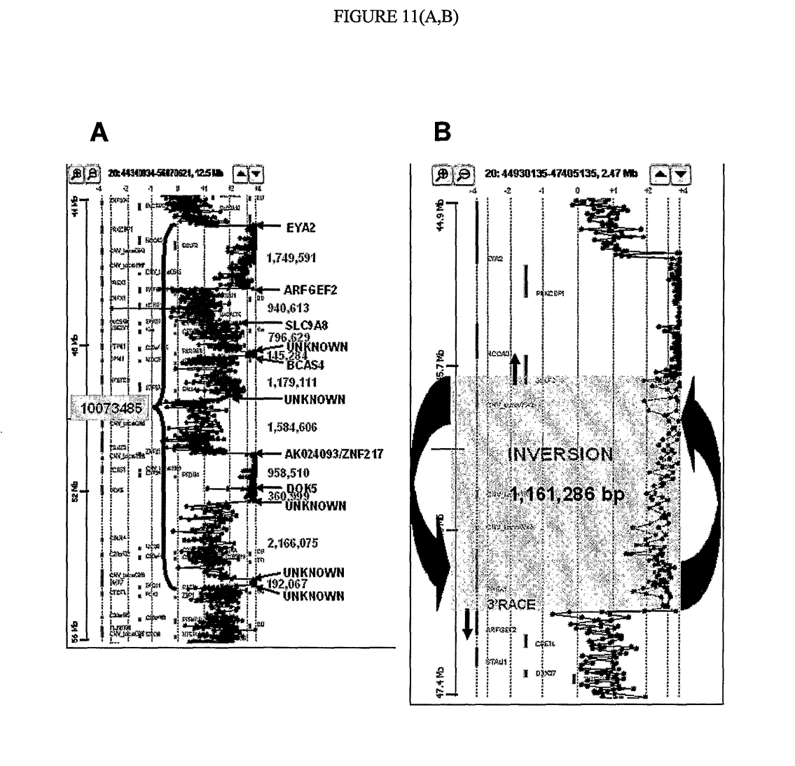

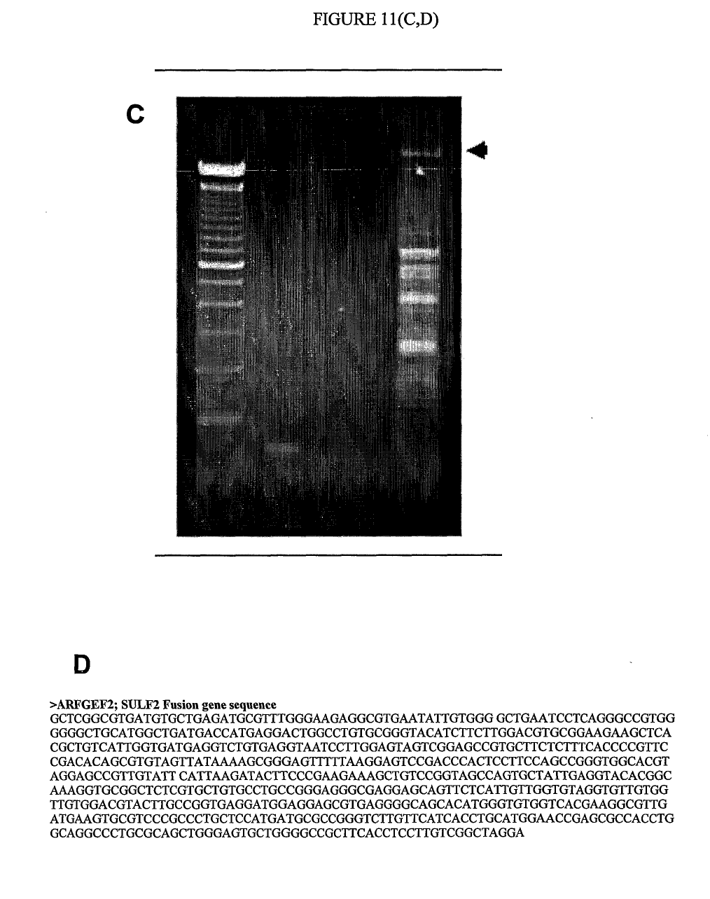

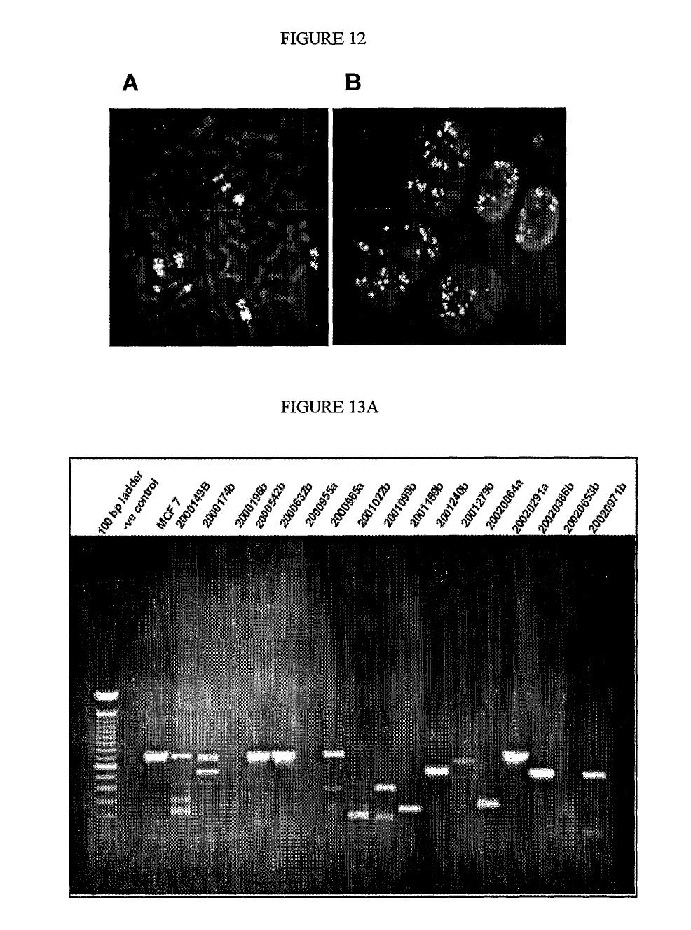

Among the 83 CNT region identified within genes, genes from the commonly amplified region in breast cancer were selected. Amplification at 20q13 reported in 20-39% of primary breast cancer is known to be associated with aggressive clinical behaviour. A non-contiguous amplification of a 10 mb region at 20q13 identified nine CNT regions affecting EYA2, ARFGEF2, SLC9A8, BCAS4, ZNF217 and DOK5 genes and three in intergenic regions (FIG. 11A). In our further validation of other CNT regions, the present inventors found one of the CNT located between 46972419 and 46978778 by with 6,359 by genomic intervals indicated a rearrangement in intron 1 of ARFGEF2 gene. 3′ RACE from exon 1 amplified a 2.7 kb fragment (FIG. 11C) containing the first exon of ARFGEF2 fused with third exon of SULF2 located at about 1.1 mb upstream of ARFGEF2. The genomic organization of ARFGEF2 and SULF2 genes on the plus and minus strand, respectively, indicates an inversion event within the 1.1 mb resulting in the formation of fusion gene (FIG. 11B). The current studies further indicate that many such sub microscopic rearrangements within amplified regions might affect many other genes within amplicons. The FISH analysis using BAC clones RP11-644F19 (ARFGEF2) and RP11-1133B15 (SULF2), formed co localizing signals confirming the fusion of ARFGEF2 and SULF2 genes (FIG. 12). This is the first report to show the isolation of a novel fusion gene from a CNT region by high-resolution analysis of an amplicon. The complex rearrangements within an amplicon indicate that the other genes within an amplicon, without a valid CNT, also might undergo rearrangement and possibly producing a fusion gene.

### Recurrent Fusion of ARFGEF2/SULF2 Genes in Breast Cancer

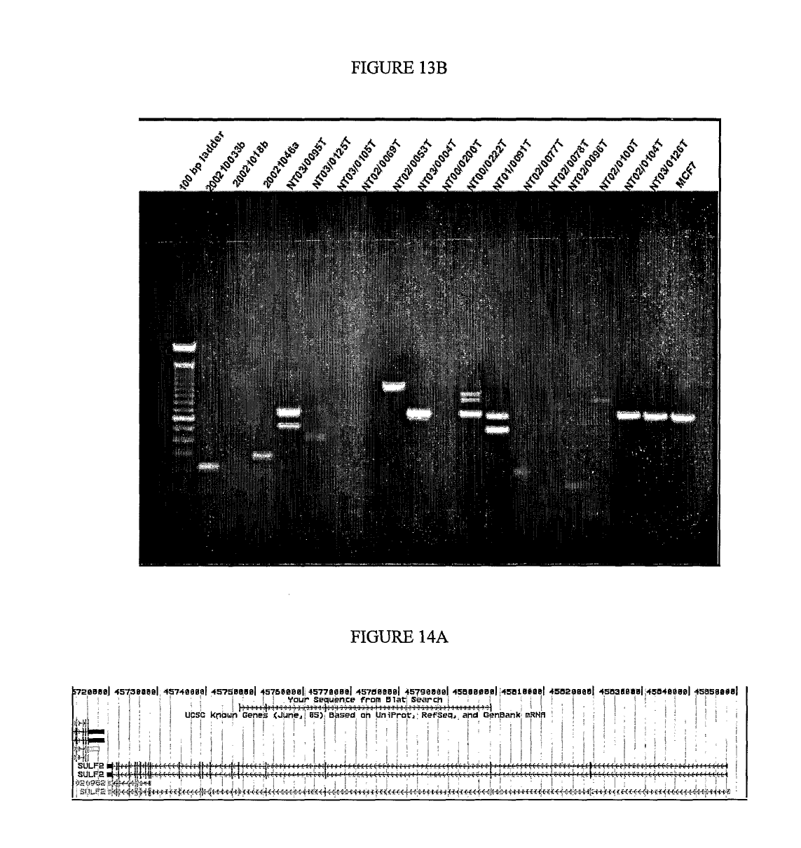

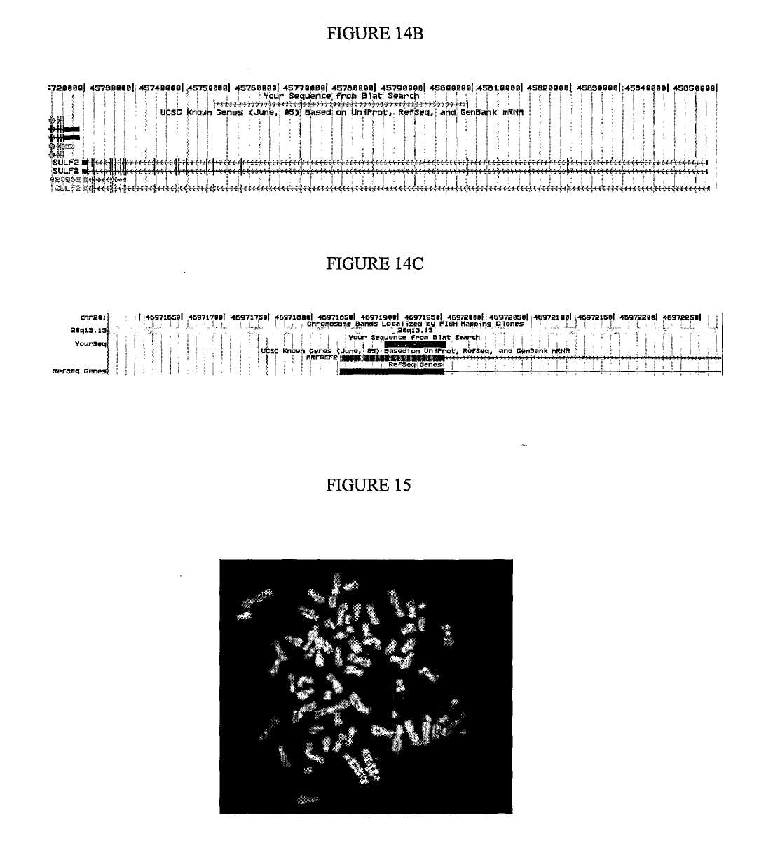

Further to the confirmation of ARFGEF2/SULF2 fusion gene in MCF7, the present inventors extended our analysis to estimate the incidence in primary breast cancer tumors and breast cancer cell lines. RT PCR analysis using the following primers from ARFGEF2 exon 1 (5′ TAGCCGACAAGGTGAAG 3′) (SEQ ID NO: 6) and reverse primer from exon 6 of SULF2 gene (5′ GTGTAGCGCATGATCCAGTG 3′) (SEQ ID NO: 7) showed the presence of fusion gene in 17/35 (49%) of primary tumors (FIG. 13) and none of the 11 cell lines were positive. Of the 17 cases positive by RT PCR, 11 cases showed the band corresponding to the size amplified in MCF7, three cases showed a small second band in addition to the first band and three cases showed only the small band. Sequence analysis confirmed fusion in all the cases and the second small band is a variant fusion gene containing all exons except exon 5 of SULF2 gene (FIG. 14B). The results indicate that high resolution view of an amplicon is detected using low-resolution CGH methods. This study has also Identified contiguous genomic amplifications producing distinct CNT regions and suggests that segmental amplification produce many CNT affecting known genes. Since amplified regions are rich source of genomic rearrangements they have the ability to produce novel fusion genes. Further as ARFGEF2 is a recurrent fusion gene found in a large number of breast cancer tumors this indicates that it serves as a new molecular marker for this type of cancer.

### Recurrent Promiscuous Rearrangement of RPS6 KB1 Gene

Gene 3: RPS6 KB1/TMEM49 (SEQ ID NO: 17) ins(17)(q23.2)
Isolation of Promiscuous Fusion Gene Produced by Insertion and Inversion within an Amplicon.

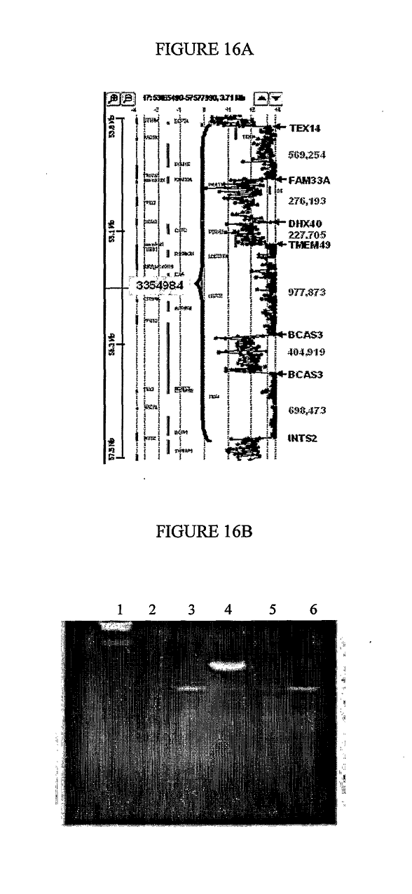

With the successful cloning of a fusion gene from 20q13 amplicon, the present inventors extended our analysis to the non contiguous amplification of about 3.3 mb at 17q23 containing seven CNT regions affecting TEX14, FAM33A, DHX40, TMEM49, INTS2 genes and BCAS3 gene with two CNT regions (FIG. 16, A). Three fusion genes BCAS4/BCAS3, BCAS3/ATXN7 (SEQ ID NO: 19), and RPS6 Kb1/TMEM49 (SEQ ID NO: 17), were identified within this amplicon and isolated. RPS6 Kb1 and TMEM49 genes are located 52 kb apart at 17q23 within the 3.3 mb amplicon. A CNT region identified at the 3′end of TMEM49 starting at 5′ 55260272 to 55262899 3′ with a genomic interval of 2627 bp. Among all the CNT regions in MCF7 within genes, this is the smallest genomic interval identified in TMEM49. Although RPS6 Kb1 gene did not contain a CNT region, it is well within a highly amplified region distributed to many locations in MCF7 genome, as confirmed by FISH analysis (FIG. 15). Based on this observation, analysis of MCF7 transcriptome by paired end ditag method (Ruan et al, 2007) showed a Tag0 cluster with 5′ tag correspond to RPS6 KB1 and 3′ tag correspond to TMEM49. Initially we performed RT PCR analysis using RPS6 KB1 forward primer (5′GCTGAAC TTTAGGAGCCAG3′) (SEQ ID NO: 8) and TMEM49 reverse primer (5′TTTTCCTCCCAAGCAAAACA3′) (SEQ ID NO: 9) amplified a 1.2 kb PCR product. Sequence analysis confirmed fusion of first four exons of RPS6 KbB1 with the last exon of TMEM49. This observation independently validated in the cloning and sequencing group in GIS and reported in a recent publication (Ruan et al., 2007). We further confirmed this finding by 3′RACE PCR using primers from the first exon of RPS6 KB1 (FIG. 16, B) which amplified a similar size product. The present inventors extended the validation study to estimate the incidence of this fusion gene and performed RT PCR screening in 11 breast cancer cell lines and 35 primary breast cancer tumors. In all the samples a PCR product corresponding to the normal transcript was amplified but none of the samples were positive for RPS6 Kb1/TMEM49 fusion gene. Rearrangement of RPS6 KB1 without an obvious CNT, and the presence of RPS6 KB1 sequence at multiple locations as revealed by FISH indicates that the genes within an amplicon undergoes rearrangement to form fusion genes but not necessarily with the same partner genes in all the samples. In order to confirm the possibility of promiscuous rearrangement of RPS6 Kb1 further evaluation of RPS6 KB1 gene by 3′ RACE PCR instead of RT PCR was done. A new breakpoint in RPS6 KB1 gene fused with a partner gene other than TMEM49 was identified. Sequence alignment of first four exons of RPS6 Kb1 with the last exon of TMEM49 in BLAST analysis represents the alignment of the RPS6 Kb1/TMEM49 fusion gene (SEQ ID NO: 17).

The kinase domain of RPS6 KB1 gene is partially preserved in the fusion gene and no coding sequences from TMEM49 is involved in the fusion transcript. Due the close proximity of the presence of mir-21, this translocation may be targeted to the over expression of mir 21. Activation of mir-21 by a protein kinase is a new avenue for future research, as it has been known that majority of the microRNA genes are located in chromosomal breakpoints frequently rearranged in cancer. It is also important to note that microRNA (mir-21) is located 245 bp telomeric to the last untranslated exon of TMEM49 gene and 51745 by upstream from the first exon of RPS6 KB1. Mir-21 is reported to be over expressed in breast cancer and glioblastoma.

Since the fusion gene contains only the last untranslated exon of TMEM49, this study indicates that, in addition to the formation of RPS6 KB1/TMEM49 fusion gene, this translocation is targeted to the over expression of mir-21.

Gene 4: RPS6 Kb1/EAP30 inv(17)(q23.2-q21.32)

### Promiscuous Rearrangement of RPS6 KB1 Detected by 3′RACE

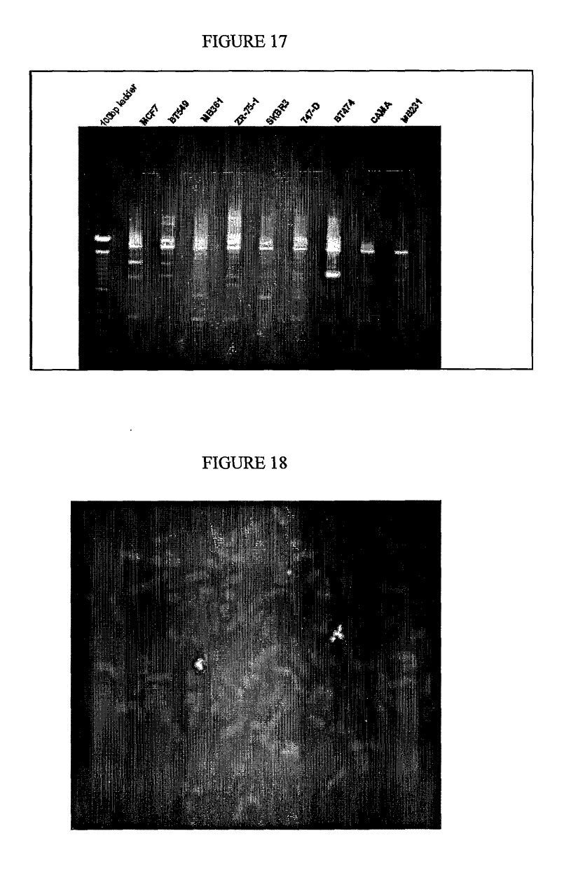

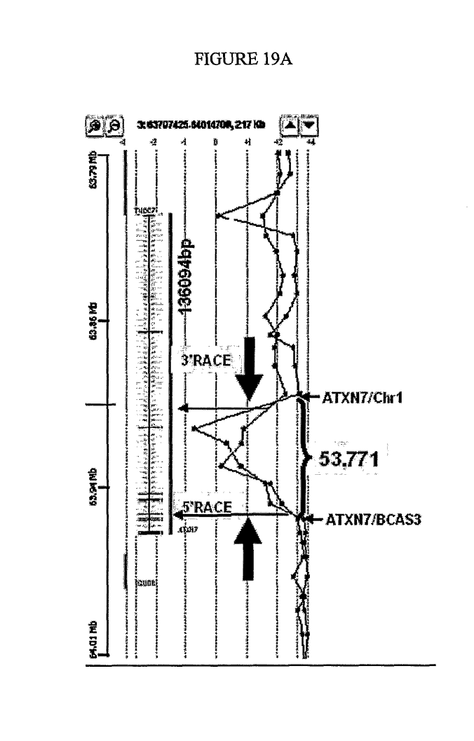

As discussed in the previous section, the distribution of amplified sequences of RPS6 Kb1 to many locations in MCF7 genome suggested a possibility of promiscuous rearrangement within in the amplified sequences. 3′RACE PCR from the first exon of RPS6 KB1 revealed the presence of normal RPS6 KB1 transcript in all the cell lines and primary breast tumours. In BT474 cell line a second band of about 900 bp showed (FIG. 17 A, B) fusion of first exon of RPS6 KB1 with the second exon of EAP30 (SNF8) gene located about 10 mb upstream in the opposite orientation indicating an inversion within the amplified region resulted in the fusion similar to the ARFGEF2/SULF2 (SEQ ID NO: 16) fusion identified at 20q13. The present inventors validated their finding by RT PCR and FISH analysis using BAC clones RP11-111G18 from 5′ end of RPS6 Kb1 and RP11-622D16 from 3′ end of EAP30 genes. FISH analysis confirmed co localization of both genes on a rearranged chromosome. In BT474 the amplified sequences are located on the same chromosome (FIG. 18). The formation of ARFGEF2/SULF2 (SEQ ID NO: 16) and RPS6 Kb1/EAP30 fusion genes by inversion within an amplified region indicates that the genes within an amplicon even without an obvious CNT undergo rearrangement to form novel fusion genes. Sequence alignment of first exon of RPS6 Kb1 with exons 2-9 of EAP30 in BLAST analysis represents the alignment of the RPS6 Kb1/EAP30 fusion gene.

Isolation of Two Fusion Genes from Two CNT Regions within a Gene

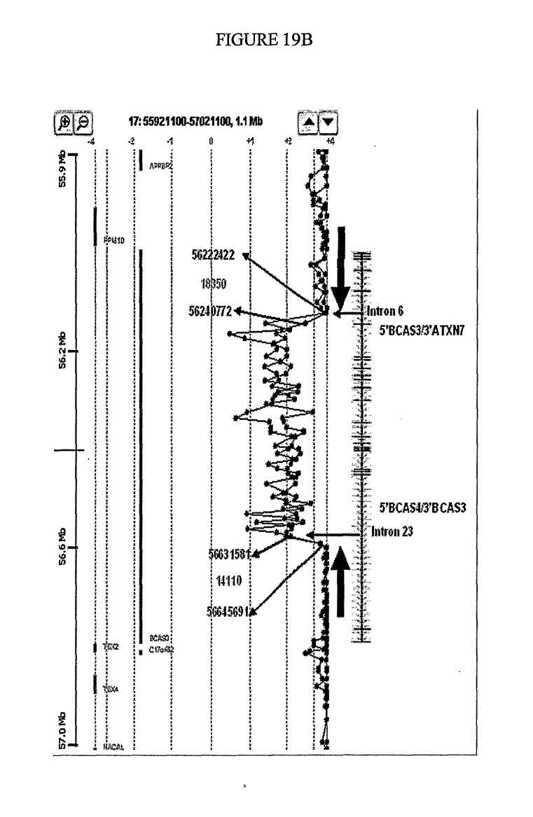

Among the 83 genes identified to contain CNT regions, BCAS3 and ATXN7 genes showed two CNT regions formed by high level amplification of small regions at the 3′ and 5′ ends and a segment in between amplified at a low level (FIG. 19 A, B).

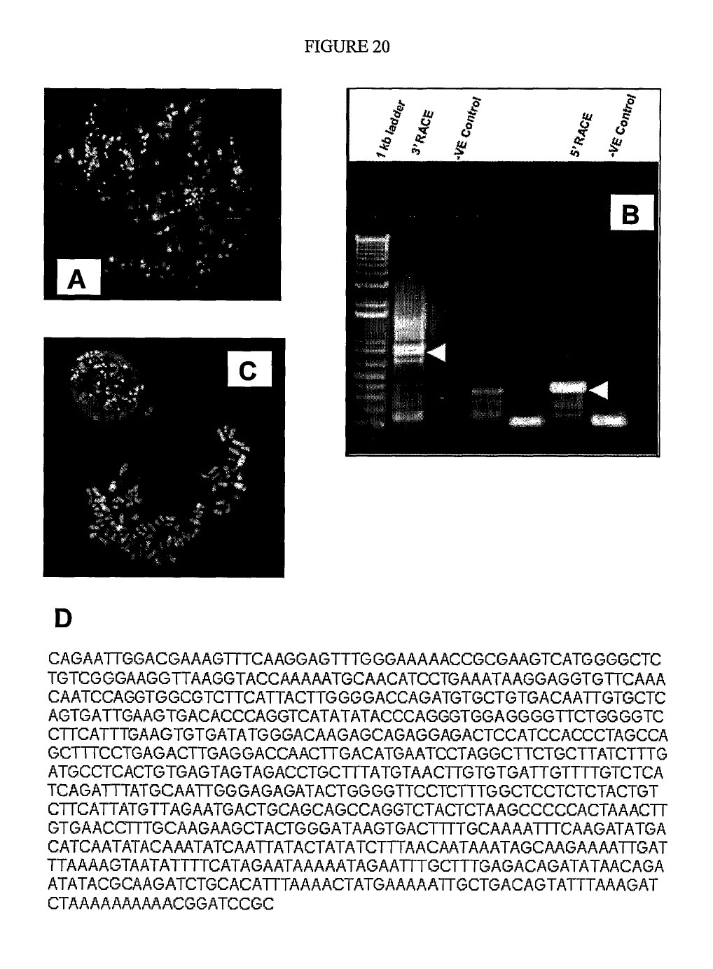

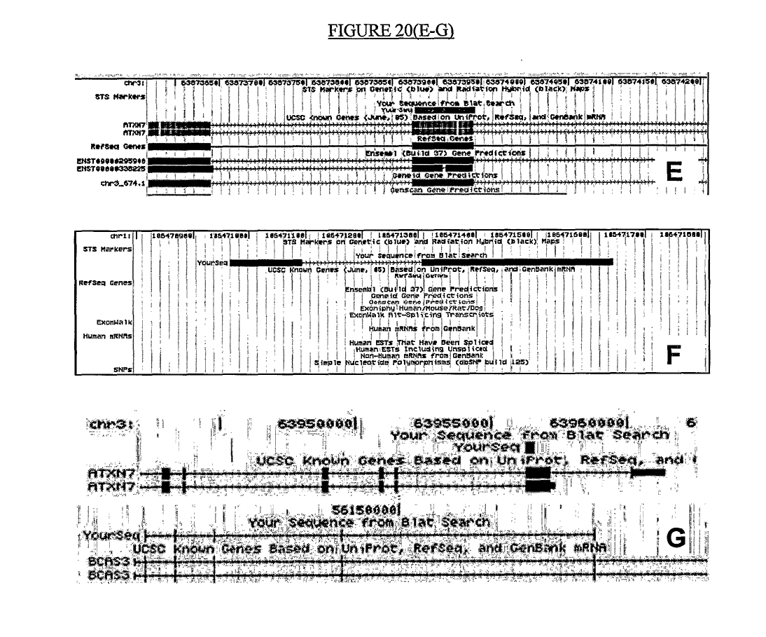

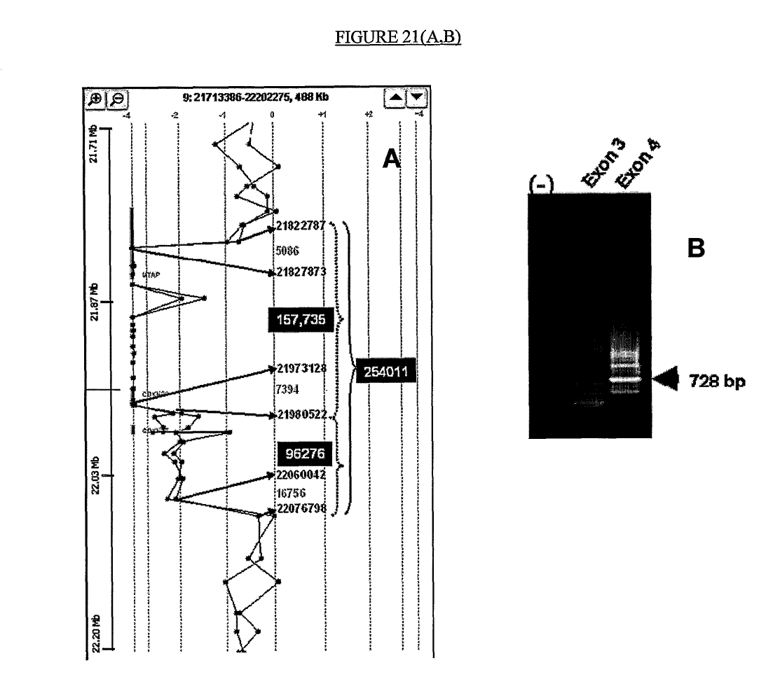

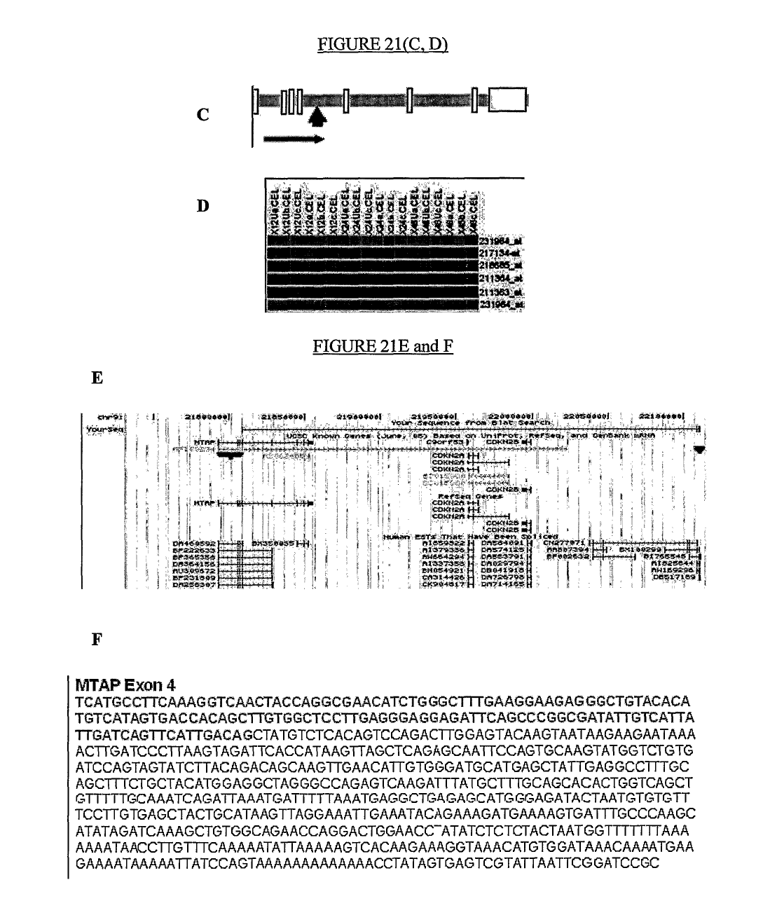

Genes 5 and 6: ATXN7/Novel gene of SEQ ID NO:1 (SEQ ID NO: 18) t(1; 3)(p21.1; 14.1) and BCAS3/ATXN7 (SEQ ID NO: 19) t(3; 17)(q23.2; p21.1). ATXN7 gene is located on chromosome 3 at genomic interval from 63,825,273 bp to 63,961,367 bp. In MCF7, an amplification of 3.35 mb starting from 5′61579369 to 649377253′ include ATXN7 in which a small region of 53,771 by region starting from 5′63901813 to 639555843′ is not amplified at the same level as the rest of the 5′ and 3′ end of ATXN7 gene resulting in the formation of two distinct CNT regions leaving exons 1-4 at the 5 end and exons 11 and 12 at the 3′end. FISH analysis using BAC clone RP11-1143K18 showed insertion of ATXN7 sequences at multiple locations in the genome (FIG. 20, A). The present inventors performed 3′ and 5′ RACE using the following primers; 3′RACE 5′CTGAAGTGATGCTGGGACAGT3′ (SEQ ID NO: 10), from exon 3 and a nested primer 5′ACAGAATTGGACGAAAGTTTCAA3′ from exon 4 (SEQ ID NO: 11) and 5′ RACE using primers from exon 12 (5′GGTACTGCTACTGGCATTTTGAC3′) (SEQ ID NO: 12) and a nested primer 5′ATTTGCTGGATTTCAATTTCTGA3′ from exon12 (SEQ ID NO: 13). Interestingly, both RACE PCR reactions amplified distinct PCR products (FIG. 20B). Sequence analysis of 3′ RACE product identified fusion of ATXN7 with a novel gene (SEQ ID NO: 1) on chromosome 1p21 (FIG. 20C) and 5′ RACE product identified fusion of 3′ end of ATXN7 with exon 6 of BCAS3 gene at 17q23.2. FISH analysis using BAC clones RP11-1143K18 (AXTN7) and RP11-1081E4-BCAS3 5′ confirmed both amplification and fusion (FIG. 20C). Of the two CNT regions in BCAS3 gene the 5′ CNT region is located in intron 6 leaving the first 6 exons fused with ATXN7. The 3′ CNT in BCAS3 found at intron 23 of BCAS3 leaving the last two exons fused with BCAS4. This rare occurrence of two rearrangements within a gene resulting in the formation of two distinct fusion genes is an important observation not descried before. This is the first study showing sub microscopic rearrangement associated with unbalanced copy number changes.

Novel Fusion Gene Isolated from a CNT Region in the Commonly Deleted Region in Multiple Cancer Types
Gene 7: MTAP/Novel gene of SEQ ID NO: 2 (SEQ ID NO: 20) (del (9)(p21)

Large genomic deletions are common in a variety of cancer types. Deletions at 9p21 has been reported in variety of cancer types including gliomas, mesothelioma, childhood, ALL, lung cancer and leukemia confirmed by FISH and other molecular methods. The extent of the deleted region is quite variable in different samples however a recurrent deletion boundary spanning intron 4 was reported (Batova et al., 1996). Although the genes located within the deletion are considered to be lost depending on the extent of the deletion, but it is intriguing to note that the boundaries of deletion might fall within known genes forming a distinct CNT region. The present inventors observed a CNT within MTAP gene in region of 254 kb deletion including part of MTAP gene starting in intron 4 and CDKN2A and CDKN2B genes leaving the first 4 exons of MTAP genes intact with at least one copy. We applied our nested RACE PCR strategy using primers from exon 4 (5′ATCATGCCTTCAAAGGTCAACTA3′) (SEQ ID NO: 14) and performed 3′RACE and found a 728 by PCR product of a fusion gene containing the first four exons of MTAP gene and an EST sequence from the immediately flanking region of the deletion at the 5′ end of the deletion suggesting the formation of an frame fusion following the deletion event. Gene expression data for all the probes included for genes within the deleted region including MTAP gene showed no expression due to the fact that all the isolation of a novel fusion gene (SEQ ID NO: 2) from a region commonly deleted in a variety of cancer types.

### CONCLUSION

Analysis of array CGH data from MCF7 cell line showed more than 100 regions of copy number gains and losses, ranging in the size from 30 kb to 30 MB. These include regions with low level copy number gains, losses and high level amplifications (3 to >40 copies). In addition to the identification of regions of gains and losses, careful analysis at the copy number transition boundaries revealed 124 breakpoints within known and cancer related genes. Of the 124 breakpoints, 33% of breakpoints occurred at the intergenic regions and 67% identified within genes at either 3′ or 5′ end providing a direct clue to map the breakpoint in a gene within a small genomic distance. Further, it underscores the importance of the concentration of breakpoints within genes rather than random breaks within intergenic regions. This indicates that most, if not all, the rearrangements are targeted to affect the function of genes either by dysregulation or formation of fusion genes. Therefore, this study is a conceptual jump in understanding, the unbalanced copy number changes in solid tumor genome by providing a methodological approach to discover novel fusion genes.

This invention allows identifying novel fusion genes by analyzing unbalanced copy number changes in various cancer types using array CGH technology since existing technologies for genome characterization suffer from its own limitations, for example, BAC, cDNA and low density tiling arrays do not provide sufficient resolution to identify copy number transition with in a short genomic interval. Other methods including End sequence profiling (ESP), representation oligonucleotide microarray (ROMA) detects rearrangements at large genomic interval (>100 kb). The array designs used in this study identified start and stop position of breakpoint intervals at a resolution as low as 2.7 kb to maximum of 23 kb (Table 1).

### REFERENCES

1. Batova A, Diccianni M B, Nobori T, Vu T, Yu J, Bridgeman L, Yu A L. Frequent deletion in the methylthioadenosine phosphorylase gene in T-cell acute lymphoblastic leukemia: strategies for enzyme-targeted therapy. Blood. 1996 Oct. 15; 88(8):3083-90.
2. Chan J A, Krichevsky A M, Kosik K S. MicroRNA-21 is an antiapoptotic factor in human glioblastoma cells. Cancer Res. 2005 Jul. 15; 65(14):6029-33.
3. Iorio M V, Ferracin M, Liu C G, Veronese A, Spizzo R, Sabbioni S, Magri E, Pedriali M, Fabbri M, Campiglio M, Menard S, Palazzo J P, Rosenberg A, Musiani P, Volinia S, Nenci I, Calin G A, Querzoli P, Negrini M, Croce C M. MicroRNA gene expression deregulation in human breast cancer. Cancer Res. 2005 Aug. 15; 65(16):7065-70.
4. Mitelman F, Johansson B, Mertens F. The impact of translocations and gene fusions on cancer causation. Nat Rev Cancer. 2007 April; 7(4):233-45. Epub 2007 Mar. 15. Review
5. Ruan Y, Ooi H S, Choo S W, Chiu K P, Zhao X D, Srinivasan K G, Yao F, Choo C Y, Liu J, Ariyaratne P, Bin W G, Kuznetsov V A, Shahab A, Sung W K, Bourque G, Palanisamy N, Wei C L. Fusion transcripts and transcribed retrotransposed loci discovered through comprehensive transcriptome analysis using Paired-End diTags (PETs). Genome Res. 2007 June; 17(6):828-38.
6. Sambrook and Russell; 2001. Molecular cloning: A Laboratory manual, Cold Spring Harbour Laboratory press, New York.
7. Tomlins S A, Rhodes D R, Perner S, Dhanasekaran S M, Mehra R, Sun X W, Varambally S, Cao X, Tchinda J, Kuefer R, Lee C, Montie J E, Shah R B, Pienta K J, Rubin M A, Chinnaiyan A M. Recurrent fusion of TMPRSS2 and ETS transcription factor genes in prostate cancer. Science. 2005 Oct. 28; 310(5748):644-8.

1. An isolated fused gene comprising at least one first gene and/or fragment thereof fused to at least one second gene and/or fragment thereof, wherein at least the first and/or the second gene, independently, is selected from the group consisting of: RCC2, CENPF, ARFGEF2, SULF2, MTAP, ATXN7, BCAS3, RPS6 KB1, TMEM49, EAP30, a gene having the nucleotide sequence SEQ ID NO:1, and a gene having the nucleic acid SEQ ID NO:2, or a fragment thereof.

2. The fused gene according to claim 1, wherein the first gene is selected from the group consisting of: RCC2, ARFGEF2, MTAP, ATXN7, BCAS3, and RPS6 KB1, or a fragment thereof.

3. The fused gene according to any one of the preceding claims, wherein the second gene is selected from the group consisting of: CENPF, SULF2, a gene having the nucleotide sequence SEQ ID NO:1, a gene having the nucleotide sequence of SEQ ID NO:2, ATXN7, TMEM49, and EAP30, or a fragment thereof.

4. The fused gene according to any one of the preceding claims, wherein the first and/or the second gene is ATXN7.

5. The fused gene according to any one of the preceding claims, wherein the first and/or the second gene is ARFGEF2.

6. The fused gene according to any one of the preceding claims, wherein the first and/or the second gene is SULF2.

7. The fused gene according to any one of the preceding claims, wherein the first and/or second gene is RPS6 KB1.

8. The fused gene according to any one of the preceding claims, wherein the first and/or second gene is a gene comprising the nucleotide sequence SEQ ID NO:1 or SEQ ID NO:2 or a fragment thereof.

9. The fused gene according to any one of the preceding claims, wherein the fusion is by genomic translocation, insertion, inversion, amplification and/or deletion.

10. The fused gene according to any one of the preceding claims, wherein the fused gene is selected from the group of fused genes RCC2/CENPF, ARFGEF2/SULF2, ATXN7/a gene comprising the nucleotide sequence SEQ ID NO:1, MTAP/a gene comprising the nucleotide sequence SEQ ID. NO:2, BCAS3/ATXN7, RPS6 KB1/TMEM49, and RPS6 KB1/EAP30, or fragments) thereof.

11. The fused gene according to any of the preceding claims, wherein the fused gene is ARFGEF2/SULF2 fusion gene comprising the nucleic acid sequence of SEQ ID NO: 16 and/or a fragment thereof.

12. The fused gene according to any of the preceding claims, wherein the fused gene is RPS6 KB1/TMEM49 fusion gene comprising the nucleic acid sequence of SEQ ID NO: 17 and/or a fragment thereof.

13. The fused gene according to any of the preceding claims, wherein the fused gene is ATXN7/a gene having the nucleotide sequence SEQ ID NO:1 gene fusion comprising the nucleic acid sequence of SEQ ID NO: 18 and/or a fragment thereof.

14. The fused gene according to any of the preceding claims, wherein the fused gene is ATXN7/BCAS3 fusion gene comprising the nucleic acid sequence of SEQ ID NO: 19 and/or a fragment thereof.

15. The fused gene according to any of the preceding claims, wherein the fused gene is MTAP /a gene having the nucleotide sequence SEQ ID NO:2 gene fusion comprising the nucleic acid sequence of SEQ ID NO: 20 and/or a fragment thereof.

16. A vector comprising the fused gene according to any one of the preceding claims.

17. An isolated nucleic acid comprising the nucleotide sequence SEQ ID NO:1 and/or SEQ ID NO:2, or a fragment thereof.

18. A vector comprising the isolated nucleic acid according to claim 17.

19. A diagnostic and/or prognostic kit for the diagnosis and/or prognosis of tumour in a subject comprising detecting at least one fused gene, according to any one of the claims 1 to 15, wherein the presence of the fused gene is indicative of presence and/or the stage of tumour.

20. The diagnostic and/or prognostic kit according to claim 19, wherein the kit comprises at least one nucleic acid molecule capable of hybridizing to and/or complementary to the fused gene and/or a fragment thereof, wherein hybridization is indicative of presence and/or the stage of tumour.

21. A diagnostic and/or prognostic kit for the diagnosis and/or prognosis of tumour in a subject, wherein the kit comprises one or more fragment representative of a genome capable of hybridizing to differentially labelled genomic DNA isolated from tumour tissue from at least one subject and from a control tissue, wherein an increase or decrease of the hybridization intensity and/or signal(s) of the label in the tumour tissue, compared to that in control tissue detects copy number transition (CNT) regions in the tumour tissue, indicative of presence and/or stage of tumour.

22. The diagnostic and/or prognostic kit according to claim 21, wherein the CNT regions comprise fused gene(s).

23. The diagnostic and/or prognostic kit according to claim 22, wherein the fused genes are detected by FISH and/or RACE technique.

24. The diagnostic and/or prognostic kit according to claim 22 or 23, wherein the fused gene is at least one fused gene according to claims 1 to 15.

25. The diagnostic and/or prognostic kit according to claims 19 to 24, wherein the tumour is stage III tumour.

26. The diagnostic and/or prognostic kit according to claims 19 to 25, wherein the tumour is solid tumour.

27. The diagnostic and/or prognostic kit according to claims 19 to 26, wherein the tumour is breast tumour.

28. A method of diagnosis and/or prognosis of presence and/or stage of tumour in a subject comprising detecting at least one fused gene, according to any one of the claims 1 to 15, wherein the presence of the fused gene is indicative of presence and/or the stage of tumour.

29. The method according to claim 28, wherein the method comprises providing at least one nucleic acid molecule capable of hybridizing to and/or complementary to the fused gene and/or a fragment thereof, wherein hybridization is indicative of presence and/or the stage of tumour.

30. A method of diagnosis and/or prognosis of presence and/or stage of tumour in a subject, wherein the method comprises providing one or more fragments representative of a genome capable of hybridizing to differentially labelled genomic DNA isolated from tumour tissue from at least one subject and from a control tissue, wherein an increase or decrease of the hybridization intensity and/or signal(s) of the label in the tumour tissue, compared to that in control tissue detects copy number transition (CNT) regions in the tumour tissue, indicative of presence and/or stage of tumour.

31. The method according to claim 30, wherein the CNT regions comprise fused gene(s).

32. The method according to claim 31, wherein the fused genes are detected by FISH and/or RACE technique.

33. The method according to claim 31 or 32, wherein the fused gene is at least one fused gene according to claims 1 to 15.

34. The method according to claims 28 to 33, wherein the tumour is stage III tumour.

35. The method according to claims 28 to 34, wherein the tumour is solid tumour.

36. The method according to claims 28 to 35, wherein the tumour is breast tumour.

37. A kit for the detecting the presence of fused genes, wherein the kit comprises one or more fragments representative of a genome capable of hybridizing to differentially labelled control and test genomic DNA wherein an increase or decrease of the hybridization intensity and/or signal(s) of test genome, compared to that in control genome detects copy number transition (CNT) regions in the test genome, wherein the CNT regions comprise fused genes.

38. The kit according to claims 37, wherein the fused gene is at least one fused gene according to claims 1 to 15.

39. A method of detecting the presence of fused genes, wherein the method comprises providing one or more fragments representative of a genome capable of hybridizing to differentially labelled control and test genomic DNA wherein an increase or decrease of the hybridization intensity and/or signal(s) of test genome, compared to that in control genome detects copy number transition (CNT) regions in the test genome, wherein the CNT regions comprise fused genes.

40. The method according to claim 39, wherein the fused gene is at least one fused gene according to claims 1 to 15.
